(19) United States
(12) Patent Application Publication
(10) Pub. No.: US 2024/0299562 A1
(43) Pub. Date: Sep. 12, 2024

(54) COMPOUNDS COMPRISING A TETRAPEPTIDIC MOIETY

(71) Applicant: COBIORES NV, Leuven (BE)

(72) Inventors: Andrea CASAZZA, Leuven (BE); Nele KINDT, Leuven (BE); Geert REYNS, Leuven (BE); Lawrence VAN HELLEPUTTE, Leuven (BE); Olivier DEFERT, Leuven (BE)

(21) Appl. No.: 18/259,040

(22) PCT Filed: Dec. 22, 2021
(86) PCT No.: PCT/EP2021/087374

§ 371 Date: Jun. 22, 2023

(30) Foreign Application Priority Data
Dec. 22, 2020 (EP) 20216764.9

(51) Int. Cl. A61K 47/64; A61P 35/00

(52) U.S. Cl. CPC A61K 47/64; A61P 35/00

(57) ABSTRACT

The present invention relates to the field of compounds intended for the treatment of cancer. Selectivity of these compounds is gained through the presence of a specific tetrapeptidic moiety allowing selective release of the drug. The drug in particular is a cytostatic, cytotoxic, or anti-cancer drug. A protective capping group can be introduced to ensure stability of the compound in blood. The tetrapeptidic moieties are ALLP or APKP.

### FIELD OF THE INVENTION

The present invention relates to the field of compounds intended for the treatment of cancer. Selectivity of these compounds is gained through the presence of a specific tetrapeptidic moiety allowing selective release of the drug. The drug in particular is a cytostatic, cytotoxic, or anti-cancer drug. A protective capping group can be introduced to ensure stability of the compound in blood. The tetrapeptidic moieties are ALLP or APKP.

### BACKGROUND OF THE INVENTION

Therapy of cancer remains one of the major challenges of medicine today. Often a combined therapeutic approach involving surgery and radiation, classical chemotoxic chemotherapy, molecular targeted drugs, or immunotherapy is required to treat cancer and/or to prevent metastasis.

A major problem in the use of chemotoxic drugs is their low selectivity for cancer cells resulting in dose limiting and life threatening toxic side effects. The most common acute toxicity is myelotoxicity resulting in a severe leukopenia and thrombocytopenia. Some of the commonly used drugs have also a more specific toxicity. Doxorubicin (Dox), an anthracycline drug, is an example of such a chemotoxic drug that induces besides severe myelotoxicity a severe cardiotoxicity. These toxicities restrict its use above a cumulative dose of 500 mg/m².

Approaches used to increase tumor specificity of a drug are conjugation with (i) a tumor-recognizing or tumor-targeting molecule (e.g. receptor ligand; see, e.g., Safavy et al. 1999-J Med Chem 42,4919-4924) or with (ii) a peptide that is cleaved preferentially in the immediate vicinity of tumor cells by proteases preferentially secreted or produced by tumor cells (“oligopeptidic prodrug”).

Tumor-specific oligopeptidic prodrugs, such as prodrugs of doxorubicin, have been developed. The prodrug-activating peptidases are not necessarily tumor specific but can increase the drug selectivity to the extent that these peptidases are (selectively) oversecreted in the extracellular space of solid tumors and play an important role in cancer cell invasion and metastasis. N-succinyl-beta-alanyl-L-leucyl-L-alanyl-L-leucyl-doxorubicin (Suc-βALAL-dox or DTS-201) was selected as such a candidate prodrug (Fernandez et al. 2001, J Med Chem 44:3750-3). Compared with unconjugated doxorubicin, this prodrug is, in mice, about 5 times, and in dogs, 3 times less toxic. Chronic treatment with Suc-βALAL-dox proved to be significantly less cardiotoxic than with Dox at doses up to 8-fold higher in rats. The improved activity of Suc-βALAL-dox over Dox was observed in several tumor xenograft models (Dubois et al. 2002, Cancer Res 62:2327-31; Ravel et al. 2008, Clin Cancer Res 14:1258-65). Two enzymes, CD10 (neprilysin or calla antigen) and thimet oligopeptidase-1 (THOP1) have been identified later in tumor cell conditioned medium and in tumor cells as activators of Suc-βALAL-dox (Pan et al. 2003, Cancer Res 63:5526-31; Dubois et al. 2006, Eur J Cancer 42:3049-56). A phase I clinical study with Suc-βALAL-dox was initiated (by DIATOS SA). Myelotoxicity by Suc-βALAL-dox occurred at three times higher doses compared with free doxorubicin. No drug-related, severe cardiac adverse events were reported, even at very high cumulative doses (2750 mg/m²). A clinical benefit was observed for 59% of evaluable patients (Delord et al., unpublished).

WO02/100353 specifically discloses chemotherapeutic prodrugs designed with a 3- to 6-amino acid oligopeptide cleavable by CD10. WO02/00263 discloses prodrugs with a 3-amino acid oligopeptide cleavable by THOP1 and at least 1 prodrug with an amino acid oligopeptide (Leu-Ala-Gly) not cleavable by CD10. WO00/33888 and WO01/95945 disclose prodrugs with a 4- to 20-amino acid oligopeptide comprising a non-genetically encoded (non-natural) amino acid at a fixed position, with said oligopeptide being cleavable by THOP1. In WO01/95945, at least 1 prodrug, with a βAla-Leu-Tyr-Leu oligopeptide, was reported to be resistant to CD10 proteolytic action. WO01/95943 discloses prodrugs with a 3- to 4-amino acid oligopeptide comprising a fixed isoleucine, said oligopeptide preferably being resistant to THOP1; no information on CD10-susceptibility or -resistance is given. A more general concept of a prodrug consisting of a drug linked to an oligopeptide (of at least 2 amino acids) itself linked to a terminal group is disclosed in WO96/05863 and was later extended in WO01/91798.

Other polymeric drug-conjugates of which the non-drug moiety is at least comprising a water-soluble polymer and a peptide (comprising 4 to 5 natural or non-natural amino acids) selectively cleavable by action of matrix metalloproteinases (MMPs) are disclosed in WO02/07770. WO02/38590, WO03/094972, WO2014/062587 and US2014/0087991 focus on anti-tumor prodrugs that are activatable by the human fibroblast activation protein (FAPα); the prodrug comprises an oligopeptide of 4 to 9 amino acids with a cyclic amino acid at a fixed position. WO99/28345 discloses prodrugs that are proteolytically cleavable by prostate-specific antigen (PSA) in the oligopeptide of less than 10 amino acids present in the prodrug.

WO97/34927 revealed the FAPα-scissable prodrugs Ala-Pro-7-amino-4-trifluoromethylcoumarin and Lys-Pro-7-amino-4-trifluoromethylcoumarin. WO00/71571 focuses on FAPα-scissable prodrugs, with some further experimental investigations on proteolytic sensitivity to CD26 (dipeptidylpeptidase IV). Other prodrugs activatable by FAPα include prodrugs of the promellitin toxin (LeBeau et al. 2009, Mol Cancer Ther 8, 1378-1386), prodrugs of doxorubicin (Huang et al. 2011, J Drug Target 19, 487-496), prodrugs of thapsigargin (Brennen et al. 2012, J Natl Cancer Inst 104, 1320-1334), and prodrugs comprising an oligopeptide of 4 to 9 amino acids with a cyclic amino acid at a fixed position (WO03/094972).

WO01/68145 discloses MMP-cleavable but neprilysin (CD10)-resistant doxorubicin prodrugs (see Example 1001 therein) comprising a 3- to 8-amino acid oligopeptide. Metalloproteinase- and plasmin-sensitive doxorubicin prodrugs have been developed, as well as CNGRC-peptide conjugates with doxorubicin (Hu et al. 2010, Bioorg Med Chem Lett 20, 853-856; Chakravarty et al. 1983, J Med Chem 26, 638-644; Devy et al. 2004, FASEB J 18, 565-567; Vanhensbergen et al. 2002, Biochem Pharmacol 63, 897-908).

WO97/12624, WO97/14416, WO98/10651, WO98/18493 and WO99/02175 disclose peptide-comprising prodrugs wherein the peptide is cleavable by the prostate-specific antigen (PSA).

WO2014/102312 describes prodrugs comprising tetrapeptides that are cleaved in 2 steps by at least 2 different peptidases enriched in the vicinity of tumor cells. Such 2-step activation increased drug selectivity. Disclosed tetrapeptides include ALGP, KLGP and TSGP.

Common to all above prodrugs is the presence of a protecting or capping moiety, usually covalently linked to the N-terminal side of the oligopeptide, which adds to the stability of the prodrug and/or adds to the prevention of internalization of the prodrug into a cell such as a target cell. Such protecting or capping moieties include non-natural amino acids, ß-alanyl or succinyl groups (e.g. WO96/05863, U.S. Pat. No. 5,962,216). Further stabilizing, protecting or capping moieties include diglycolic acid, maleic acid, pyroglutamic acid, glutaric acid, (e.g., WO00/33888), a carboxylic acid, adipic acid, phthalic acid, fumaric acid, naphthalene dicarboxylic acid, 1,8-naphtyldicarboxylic acid, aconitic acid, carboxycinnamic acid, triazole dicarboxylic acid, butane disulfonic acid, polyethylene glycol (PEG) or an analog thereof (e.g., WO01/95945), acetic acid, 1- or 2-naphthylcarboxylic acid, gluconic acid, 4-carboxyphenyl boronic acid, polyethylene glycolic acid, nipecotic acid, and isonipecotic acid (e.g., WO02/00263, WO02/100353), succinylated polyethylene glycol (e.g., WO01/91798). A new type of protecting or capping moiety was introduced in WO2008/120098, being a 1,2,3,4 cyclobutanetetracarboxylic acid. The protecting or capping moiety in WO02/07770 may be polyglutamic acid, carboxylated dextranes, carboxylated polyethylene glycol or a polymer based on hydroxyprolyl-methacrylamide or N-(2-hydroxyprolyl)methacryloylamide. WO2014/102312 introduced phosphonoacetyl-, and further used the previously known succinyl group, as a capping group or capping moiety.

### SUMMARY OF THE INVENTION

The invention relates to compounds having the general structure C-OP-D, wherein: C is a capping group; OP is a tetrapeptidic moiety selected from the group consisting of ALLP (SEQ ID NO:1) and APKP (SEQ ID NO:2); D is a drug; or a pharmaceutically acceptable salt of said compound, a pharmaceutically acceptable crystal or co-crystal comprising said compound, or a pharmaceutically acceptable polymorph, isomer, or amorphous form of said compound. In one embodiment, said drug D is a cytotoxic drug, a cytostatic drug, or is an anti-cancer drug. In another embodiment, the linkage between OP and D is direct, or is indirect via a linker or spacing group. In a further embodiment, the linkage between C and OP is direct, or is indirect via a linker or spacing group. In yet a further embodiment, both linkage between OP and D and linkage between C and OP are direct, or are indirect via a linker or spacing group. In a specific embodiment, such linker or spacing group is a self-eliminating linker or spacing group. In a further embodiment, any of the above compounds, salt, crystal, co-crystal, polymorph, isomer or amorphous form thereof, is further complexed with a macrocyclic moiety.

The invention further relates to compositions comprising any one of the compound, salt thereof, crystal thereof, co-crystal comprising it, or polymorph, isomer or amorphous form of said compound. Such composition may further comprise at least one of a pharmaceutically acceptable solvent, diluent or carrier, such as to form e.g. a pharmaceutically acceptable composition.

The invention further relates to (compositions comprising any one of) the compound, salt thereof, crystal thereof, co-crystal comprising it, or polymorph, isomer or amorphous form of said compound for use as a medicament or for use in the manufacture of a medicament; such as for use in (a method of) treating of a cancer or for use in the manufacture of a medicament for treating a cancer. In one embodiment, said medicament is combined with chemotherapy treatment or a combined modality chemotherapy treatment. In another embodiment, said cancer treatment is a combination chemotherapy treatment or a combined modality chemotherapy treatment. In a further embodiment, the drug moiety D of the compound C-OP-D is effective as cytotoxic, cytostatic, or anti-cancer drug in a combination chemotherapy treatment or a combined modality chemotherapy treatment.

The invention further relates to methods for synthesizing or producing any of the above compounds, said methods comprising the steps of: linking the drug D, the tetrapeptidic moiety OP, and the capping group C; wherein the linking of D, OP and C is resulting in the compound C-OP-D, and wherein the linking between drug D and tetrapeptidic moiety OP is direct or via a linker or spacing group and/or the linking between C and OP is direct, or is indirect via a linker or spacing group. Any of the production or synthesis methods may further be comprising a step of purifying the compound C-OP-D and/or comprising a step of forming a salt, amorphous form, crystal or co-crystal of the compound C-OP-D.

The invention further envisages kits comprising a container comprising the compound, salt thereof crystal thereof, co-crystal comprising it, or polymorph, isomer or amorphous form of said compound or a composition comprising any one of the foregoing.

### LEGENDS TO FIGURES

FIG. 1. Cytotoxic effect of doxorubicin and of doxorubicin-comprising compounds on colorectal cancer. (A) LS 174T and (B) HCT-116 cells were used as in vitro models for the evaluation of C-OP-D compound potency compared to the potency of the parent free drug D. Cells were seeded at a density of 15.000 cells/well (LS 174T) or 10.000 cells/well (HCT-116) and exposed to a 1-in-5 serial dilution, starting from 100 μM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 2. Cytotoxic effect of doxorubicin and of doxorubicin-comprising compounds on glioblastoma. (A) A-172 and (B) U-87 MG cells were used as in vitro models for the evaluation of C-OP-D compound potency compared to potency of the parent free drug D. Cells were seeded at a density of 7.000 cells/well and exposed to a 1-in-5 serial dilution, starting from 100 μM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 3. Cytotoxic effect of doxorubicin and of doxorubicin-comprising compounds on triple negative breast cancer. (A) MDA-MB-231 and (B) MDA-MB-468 cells were used as in vitro models for the evaluation of C-OP-D compound potency compared to the potency of the parent free drug D. Cells were seeded at a density of 10.000 cells/well and exposed to a 1-in-5 serial dilution, starting from 100 UM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 4. Cytotoxic effect of doxorubicin and of doxorubicin-comprising compounds on ovarian cancer. (A) A2780 and (B) A2780 CpR (cisplatin resistant variant of parental line A2780) cells were used as in vitro models for the evaluation of C-OP-D compound potency compared to the potency of the parent free drug D. Cells were seeded at a density of 10.000-12.000 cells/well and exposed to a 1-in-5 serial dilution, starting from 100 μM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 5. Cytotoxic effect of doxorubicin and of doxorubicin-comprising compounds on lung cancer. (A) NCI-H1299 and (B) NCI-H292 cells were used as in vitro models for the evaluation of C-OP-D compound potency compared to the potency of the parent free drug D. Cells were seeded at a density of 7.000 cells/well and exposed to a 1-in-5 serial dilution, starting from 100 UM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 6. Cytotoxic effect of doxorubicin and of doxorubicin-comprising compounds on melanoma. A2058 cells were used as an in vitro model for the evaluation of C-OP-D compound potency compared to the potency of the parent free drug D. Cells were seeded at a density of 7.000 cells/well and exposed to a 1-in-5 serial dilution, starting from 100 μM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 7. Cytotoxic effect of doxorubicin and of doxorubicin-comprising compounds on prostate cancer. DU145 cells were used as an in vitro model for the evaluation of C-OP-D compound potency compared to the potency of the parent free drug D. Cells were seeded at a density of 5.000 cells/well and exposed to a 1-in-5 serial dilution, starting from 100 μM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 8. Cytotoxic effect of doxorubicin and of doxorubicin-comprising compounds on pancreatic cancer. MIA PaCa-2 cells were used as an in vitro model for the evaluation of C-OP-D compound potency compared to the potency of the parent free drug D. Cells were seeded at a density of 10.000 cells/well and exposed to a 1-in-5 serial dilution, starting from 100 UM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 9. Cytotoxic effect of doxorubicin and of doxorubicin-comprising compounds on normal (non-cancerous) cells. Immortalized human mammary epithelial (HME-1) cells were used as an in vitro surrogate for the evaluation of C-OP-D compound toxicity towards normal tissue compared to the toxicity of the parent free drug D. Cells were seeded at a density of 10.000 cells/well and exposed to a 1-in-5 serial dilution, starting from 100 UM (PhAc-ALGP-Dox, PhAc-APKP-Dox and PhAc-ALLP-Dox) or 10 μM (doxorubicin) for 72 hrs. Cell viability was assessed using WST-1 proliferation assay. Graphs are plotted as mean±SD. Non-linear fittings from triplicate measurements were acquired according to the Sigmoidal-4PL regression model for IC₅₀ extrapolation (n=3).

FIG. 10. Cytotoxic effect of MMAE and MMAE-comprising compounds on normal (non-cancerous) cells. (A) Immortalized human mammary epithelial (HME-1) cells or (B) human umbilical vein endothelial cells (HUVEC) were used as in vitro surrogates for the evaluation of C-OP-D compound toxicity towards normal tissue compared to the toxicity of the parent free drug D. Cells were seeded at a density of 10.000 cells/well and exposed to a 1-in-5 serial dilution, starting from 500 nM for 72 hrs. Cell viability was assessed using WST-1 proliferation assay. Graphs are plotted as mean±SD. Non-linear fittings from 3-5 triplicate measurements were acquired according to the Sigmoidal-4PL regression model for IC₅₀ extrapolation (n=9-15)

FIG. 11. Cytotoxic effect of MMAE and MMAE-comprising compounds on triple negative breast cancer. MDA-MB-231 cells were used as an in vitro model for the evaluation of C-OP-D compound potency compared to the potency of the parent free drug D. Cells were seeded at a density of 10.000 cells/well and exposed to a 1-in-5 serial dilution, starting from 500 nM for 72 hrs. Cell viability was assessed using WST-1 proliferation assay. Graphs are plotted as mean±SD. Non-linear fittings from 4 triplicate measurements were acquired according to the Sigmoidal-4PL regression model for IC₅₀ extrapolation (n=12).

FIG. 12. Cytotoxic effect of MMAE and MMAE-comprising compounds on melanoma. A2058 cells were used as an in vitro model for the evaluation of C-OP-D compound potency compared to the potency of the parent free drug D. Cells were seeded at a density of 7.000 cells/well and exposed to a 1-in-5 serial dilution, starting from 500 nM for 72 hrs. Cell viability was assessed using WST-1 proliferation assay. Graphs are plotted as mean±SD. Non-linear fittings from 4 triplicate measurements were acquired according to the Sigmoidal-4PL regression model for IC₅₀ extrapolation (n=12).

FIG. 13. Cytotoxic effect of MMAE and MMAE-comprising compounds on glioblastoma. (A) A-172 and (B) U-87 MG cells were used as in vitro models for the evaluation of C-OP-D compound potency compared to the potency of the parent free drug D. Cells were seeded at a density of 7.000 cells/well and exposed to a 1-in-5 serial dilution, starting from 500 nM for 72 hrs. Cell viability was assessed using WST-1 proliferation assay. Graphs are plotted as mean±SD. Non-linear fittings from 2-4 triplicate measurements were acquired according to the Sigmoidal-4PL regression model for IC₅₀ extrapolation (n=6-12).

FIG. 14. Cell viability of hiPSC-derived astrocytes (iAstro™) after exposure to PhAc-ALGP-PABC-MMAE, PhAc-ALLP-Dox, PhAc-ALLP-PABC-MMAE, or parent free drugs. Normal astrocytes were exposed to a dose titration of C-OP-D compounds and to the parent free drugs D for 72 hrs. After measurement of Calcein-AM substrate conversion by metabolically active cells, cells were rinsed in PBS and cell viability was assessed with WST-1. (A) Dose response curve of MMAE, PhAc-ALGP-PABC-MMAE, and PhAc-ALLP-PABC-MMAE after a 10-point serial dilution (1:5) starting from 500 nM or (B) Dox and PhAc-ALLP-Dox after a 10-point serial dilution (1:5) starting from 500 μM for 4PD or 50 M for Dox. Mean±SD as Sigmoidal-4PL non-linear fitting model. n=3 replicate wells.

FIG. 15. In vivo activity of PhAc-ALLP-Dox on colorectal cancer. (A) Plot representing the volume of LS174T colorectal tumors subcutaneously implanted in Nude NMRI mice and treated with PhAc-ALLP-Dox at 10 mg/kg or 30 mg/kg, or with control vehicle (CTRL) as indicated. Mice received treatment via tail vein injection twice a week as indicated by the arrowheads. Data represents mean±SD (n=10 per group) (**** p<0.0001 versus control). (B) Percentage tumor growth inhibition (TGI (%, mean±standard deviation SD)).

FIG. 16. In vivo activity of PhAc-ALLP-PABC-MMAE on melanoma Plot representing the volume of A2058 melanoma tumors subcutaneously implanted in Nude NMRI mice and treated with MMAE, PhAc-ALLP-PABC-MMAE or with control vehicle (CTRL) as indicated. Mice received treatment via TV injection twice a week for 4 cycles as indicated by the arrowheads. Data represents mean±SD (n=9 per group).

FIG. 17. In vivo activity of PhAc-ALLP-PABC-MMAE on glioblastoma Plot representing the volume of U87 MG tumors subcutaneously implanted in Nude NMRI mice and treated with MMAE, PhAc-ALLP-PABC-MMAE or with control vehicle (CTRL) as indicated. Mice received treatment via TV injection once a week for 4 cycles as indicated by the arrowheads. Data represents mean±SD (n=8 per group).

FIG. 18. In vivo activity of PhAc-ALLP-Dox on glioblastoma Plot representing the volume of U87 MG tumors subcutaneously implanted in Nude NMRI mice and treated with Dox, PhAc-ALGP-Dox, PhAc-ALLP-Dox or with control vehicle (CTRL) as indicated. Mice received treatment via TV injection once a week for 4 cycles as indicated by the arrowheads. Data represents mean±SD (n=8 per group).

### DETAILED DESCRIPTION OF THE INVENTION

In general, the present invention describes new prodrug compounds of therapeutic agents with improved therapeutic properties, especially prodrugs comprising a therapeutic agent, in particular a therapeutic agent useful for treating a tumor or cancer. The term “prodrug” in general refers to a compound that undergoes biotransformation before exhibiting pharmacological effects. Prodrugs can thus be viewed as drugs containing specific nontoxic protective groups present in a transient manner to alter or to eliminate undesirable properties in the parent molecule (from: Vert et al. 2012, Pure Appl Chem 84:377-410). The protective groups can have one or more function such as increasing bioavailability, increasing solubility, increasing stability, avoiding or reducing premature release of the drug (thus avoiding or reducing toxicity), altering cell permeability, avoiding or reducing irritation in the subject to be treated with the drug, supporting administration of the drug to the targeted cells or organs in a subject, etc. . . . The herein described tetrapeptide-comprising compounds (also termed C-OP-D compounds, C-OP-D prodrugs, or C-OP-D prodrug compounds, or, simply compounds (according to the invention) or prodrugs (according to the invention)) were found by serendipity as being prodrugs displaying a favourable selectivity towards cancer cells (compared to healthy or non-cancer cells); the activation mechanism behind the release of the active drug moiety from these prodrugs currently remains unknown.

In one aspect, the compounds of the invention have the general structure C-OP-D, wherein:
C is a capping group;
OP is a tetrapeptidic moiety selected from (the group consisting of) ALLP (SEQ ID NO:1) and APKP (SEQ ID NO:2);
D is a drug;
a pharmaceutically acceptable salt of said compound, a pharmaceutically acceptable crystal or co-crystal comprising said compound, or a pharmaceutically acceptable polymorph or a pharmaceutically acceptable isomer of said compound.

The nature of the tetrapeptide is a key determinant of the selectivity (determined e.g. as described in the Examples hereinafter) of the above prodrug compounds, this independent of which drug is incorporated in the prodrug compound. This is demonstrated hereinafter for the tetrapeptide ALLP (SEQ ID NO:1) with prodrug compounds of doxorubicin and auristatin. Historical examples further corroborate this. For instance, Dubowchik et al. 1998 (Bioorg Med Chem Lett 8:3341-3346 and 3347-3352) and Walker et al. 2004 (Bioorg Med Chem Lett 14:4323-4327) demonstrated that once a suitable peptidic moiety has been identified, the drug D can be changed (demonstrated for doxorubicin, mitomycin C, and tallysomycin S10b). The same was illustrated for another peptidic moiety included in a prodrug: once a suitable peptidic moiety is identified, the drug D can be changed (demonstrated for doxorubicin and paclitaxel; Elsadek et al. 2010, ACS Med Chem Lett 1: 234-238, and Elsadek et al. 2010, Eur J Cancer 46:3434-3444). Such prodrugs can even be linked successfully to antibodies targeting a tumor-specific antigen (Dubowchik et al. 2002, Bioconjugate Chem 13:855-869; and Walker et al. 2004, Bioorg Med Chem Lett 14:4323-4327), or to cell-penetrating peptides (CPPs; Yoneda et al. 2008, Bioorg Med Chem Lett 18:1632-1636). In principle, moieties other than antibodies or CPPs could be coupled, such as aptamers and single domain antibodies or fragments thereof. These examples illustrate that, once a suitable peptidic moiety is identified, it can be modified at both its ends (N-terminal and C-terminal) without loosing the functionality of the identified peptidic moiety. In one embodiment, the tetrapeptidic moiety OP and the drug D in the general prodrug structure C-OP-D are directly linked (or coupled or bound) to each other, or, alternatively are linked (or coupled or bound) indirectly via a linker or spacing group. Whatever the type of linkage (or coupling or bonding), direct or indirect, the linkage should: (1) not or not significantly disturb the functionality of the tetrapeptidic moiety, i.e., should not or not significantly disturb the proteolytic scissability of OP and (2) should retain the blood stability of the compound. Determination of the functionality of a linker or spacing group in the prodrug can be tested (e.g. stability in mammalian serum, selective toxicity to cancerous cells).

### Linker or Spacing Group Between Peptidic Moiety OP and Drug Moiety D

In view of the variety of drugs that can be incorporated in a prodrug compound, a linker or spacing group (terms used interchangeably herein) can be present to create distance between the tetrapeptidic moiety and the drug moiety such as a spacer for mitigating steric hindrance in order to facilitate proteolytic or other enzymatic degradation of the tetrapeptidic moiety OP linked to the drug moiety D. Such linker or spacing group can alternatively or additionally be present to (further) increase the specificity of the prodrug compound, e.g. by providing an additional mechanism for activation of the prodrug compound or release of the drug moiety D from the C-OP-D compound. Such linker or spacing group can further alternatively or additionally be present to enable chemical linkage between the tetrapeptidic moiety and the drug moiety, i.e. the end of the linker to be connected with the drug moiety can be designed in function of chemical coupling with a suitable group present in the chemical structure of the drug moiety. A linker or spacing group may thus provide appropriate attachment chemistry between the different moieties of the C-OP-D compound (and thus providing flexibility to couple any possible drug moiety D and a tetrapeptidic moiety OP of the invention). A linker or spacing group may further alternatively or additionally be introduced to improve the synthetic process of making the C-OP-D conjugate (e.g., by pre-derivatizing the therapeutic agent or oligopeptide with the linker group before conjugation to enhance yield or specificity). A linker or spacing group may yet further alternatively or additionally be introduced to improve physical properties of the C-OP-D compound.

Although not limited thereto, such linker or spacing group may be purely self-immolative or self-eliminating by means of chemical degradation upon release of/from the tetrapeptidic moiety. Self-immolation or self-elimination of a linker or spacing group may alternatively rely on further triggers such as esterase or phosphatase activity or may rely on a redox-sensitive, pH-sensitive, etc. triggering mechanism; in the current context such linkers are likewise termed self-immolative or self-eliminating linkers or spacing groups.

The linker between OP and D can for instance be a self-immolative or self-eliminating linker or spacing group. Upon proteolytic removal of the tetrapeptidic moiety OP, such linker is spontaneously decomposing to set free the drug moiety D. The different types of self-eliminating linkers usually decompose via a spontaneous elimination or cyclization reaction. A well-known and often used self-immolative linker is p-aminobenzyloxycarbamate (PABC; alternatively p-aminobenzyloxycarbonyl) which decomposes via 1,6-benzyl elimination; o-aminobenzyloxycarbonyl (OABC) decomposes via 1,4-benzyl elimination. Linkers such as PABC are able to connect either —OH, —COOH, —NH, or —SH groups of a drug D at the one hand to the carboxy-terminal group of a tetrapeptidic moiety OP at the other hand. Substituted 3-carbamoyl-2-arylpropenal compounds are a further example of self-immolative linkers that decompose via elimination of carbamic acid; substitutions include a nitro-group, a halide (e.g. fluoride), and a methyl group (Rivault et al. 2004, Bioorg Med Chem 12:675). Self-immolative disulfide-containing linkers are a newer group of such linkers (e.g. Gund et al. 2015, Bioorg Med Chem Lett 25:122-127). An overview is also given in Table 7 of Kratz et al. 2008 (ChemMedChem 3:20-53). Such self-immolative linkers can be multimerized (e.g. dimers, trimers, . . . ) to form elongated self-immolative linkers. Such linkers can also be multimerized in the form of dendrimers potentially carrying multiple drug D moieties (e.g. Amir et al. 2003, Angew Chem Int Ed 42:4494-4499; de Groot et al. 2003, Angew Chem Int Ed 42:4490-4494).

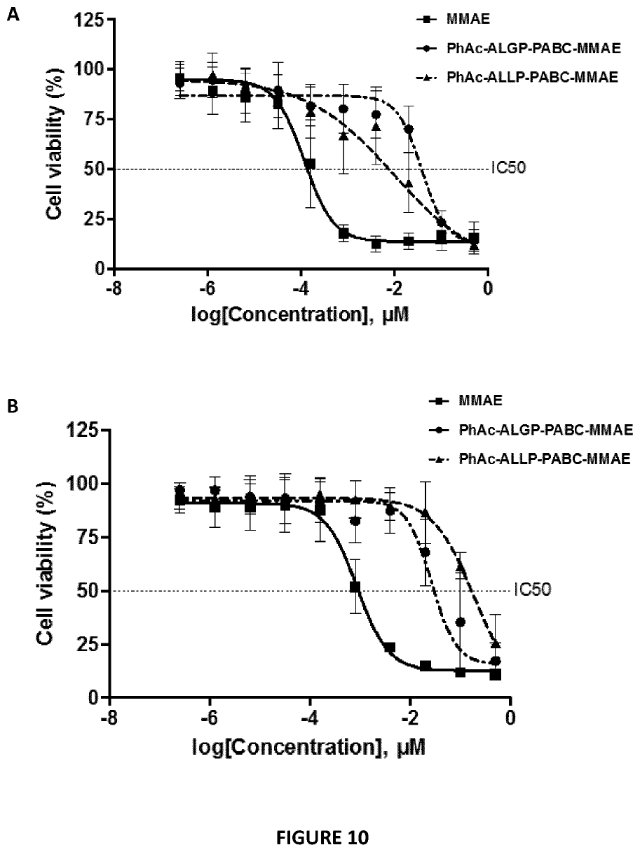

The linker between OP and D can for instance be an acid-labile linker. Taking advantage of the lower pH in the tumor environment compared to the pH in normal tissues (difference of 0.5 to 1 pH units), acid-labile linkers are preferentially cleaved in the tumor environment. Acid-labile linkers or spacers include acid-labile bonds such as carboxylic hydrazine bonds, cis-aconityl bonds, trityl bonds, acetal bonds and ketal bonds. Polymeric molecules in which the monomers are each linked to each other by an acid-labile bond are other examples of acid-labile linkers (see e.g. FIG. 10 and Table 5 of Kratz et al. 2008, ChemMedChem 3:20-53).

The linker between OP and D can for instance be a self-immolative or self-eliminating linker or spacing group wherein the self-immolation or self-elimination is occurring selectively under hypoxic/low oxygen conditions. Many tumors or cancers, in particular solid tumors or cancers, are characterized by the presence of hypoxic regions (e.g. Li et al. 2018, Angew Chem Int Ed Engl 57:11522-11531). Aromatic nitro or azido groups can be applied in this setting and reduction (in hypoxic or low oxygen areas) of these compounds starts their decomposition via 1,6- or 1,8-elimination. Analogues of nitroimidazoles, N-oxides and nitrobenzyl carbamates can be applied (e.g. imidazolylmethyl carbamates: Hay et al. 2000, Tetrahedron 56:645; e.g. nitrobenzyloxycarbonyl groups: Shyam et al. 1999, J Med Chem 42:941) and include, without limitation, 2′-(4-nitrobenzyl carbonate); 2′-(4-azidobenzyl carbonate); 2′-(4-nitrocinnamylcarbonate); 2′-O-(2,4-dinitrobenzyloxycarbonyl); 2′-O-[2-nitro-5-(allyloxycarbonyl)benzyloxycarbonyl]; 2′-O-(2-nitro-5-carboxybenzyloxycarbonyl); 2′-O-(5-methyl-nitro-1H-imidazoyl-2-yl) methyloxycarbonyl); 2′-O-(5-nitrofuran-2-ylmethyloxycarbonyl); 2′-O-(5-nitrothiophene-2-ylmethyloxycarbonyl); and 3′N-(4-azidobenzyloxycarbonyl-3′N-debenzoyl (Damen et al. 2002, Bioorg Med Chem 10:71-77; see e.g. Scheme 1 and Experimental section).

Self-elimination of a linker between OP and D can also be based on an intramolecular cyclization or lactonization reaction, such as the trimethyl lock lactonization reaction (Greenwald et al. 2000, J Med Chem 43:475-487). Such systems include, without limitation, the (alkylamino)-ethyl carbamate and [(alkylamino)ethyl]glycyl ester systems; the N-(substituted 2-hydroxyphenyl) carbamate and N-(substituted 2-hydroxypropyl) carbamate systems; and systems based on o-hydroxylphenylpropionic acid and its derivatives. These are subject of a review by Shan et al. 1997 (J Pharm Sci 86:765-767). Lactonization of coumarinic acid or its derivatives constitutes a further linker system (e.g. Wang et al. 1998, Bioorg Med Chem 6:417-426; Hershfield et al. 1973, J Am Chem Soc 95:7359-69; Lippold & Garrett 1971, J Pharm Sci 60:1019-27). Cyclization of 2′-carbamates in prodrugs is a further system leading to release of an active drug (e.g. de Groot et al. 2000, J Med Chem 43:3093-3102).

The linker between OP and D can for instance be redox-sensitive linkers susceptible to reducing conditions (such as quinones).

The linker between OP and D can for instance be a hydrophilic stopper such as a glycosylated tetra(ethylene glycol) which, upon deglycosylation (after proteolytic release of the tetrapeptidic moiety OP), spontaneous decomposes and releases the drug D (e.g. Fernandes et al. 2012, Chem Commun 48:2083-2085).

Several patents and patent applications describe other self-immolative/self-eliminating spacers, such as heterocyclic ones, releasing a drug from a targeting ligand such as an antibody have been described (e.g. U.S. Pat. No. 6,214,345; US 2003/0130189; US 2003/0096743; U.S. Pat. No. 6,759,509; US 2004/0052793; U.S. Pat. Nos. 6,218,519; 6,835,807; 6,268,488; US 2004/0018194; WO 98/13059; US 2004/0052793; U.S. Pat. Nos. 6,677,435; 5,621,002; US 2004/0121940; WO 2004/032828, US 2009/0041791). Examples of other, not necessarily self-eliminating, linker or spacer groups include aminocaproic acid, a hydrazide group, en ester group, an ether group, a sulphydryl group, ethylenediamine (or longer-CH2-chains), aminoalcohol, and ortho-phenylenediamine (1,2-diaminobenzene).

In a particular embodiment, the linker or spacer is not a self-immolative linker. Such non-self-immolative linker may still be cleavable by an enzyme present outside or inside a target cell.

In a further embodiment, the linker or spacer between the drug D and the tetrapeptide moiety OP is not comprising a proteinaceous moiety such as an L-amino acid or a derivative of an L-amino acid. In a further embodiment, said linker or spacer is not comprising a D-amino acid or a derivative of a D-amino acid. In a further embodiment, said linker or spacer is not comprising a non-natural amino acid.

In a further embodiment, the general compound structure C-OP-D described hereinabove may be complexed with a macrocyclic moiety, e.g. a self-eliminating or self-immolative macrocyclic moiety.

The self-elimination process may be a pure self-elimination process or one that is started by a further trigger (see above).

### Macrocyclic Moieties

The tetrapeptidic axle of a compound C-OP-D could further be protected by means of a macrocycle itself designed to be self-immolative or self-opening, wherein the trigger for self-immolation or self-opening could be action of an enzyme such as beta-galactosidase or beta-glucuronidase. Such macrocycle is hereinafter further termed “macrocyclic moiety”. Expression of beta-galactosidase is increased in many tumors compared to normal tissues (e.g. Chen et al. 2018, Anal Chim Acta 1033:193-198) and glucuronide prodrugs are a further class of prodrugs (e.g. Tranoy-Opalinski et al. 2014, Eur J Med Chem 74:302-313). Trapping the tetrapeptidic moiety OP of the compound of the invention in a macrocycle preferentially opening in the vicinity of tumor cells adds an additional layer of selectivity to a compound of the invention. One example of such macrocycle is a rotaxane or pseudo-rotaxane, and protection against self-opening could be through e.g. linkage with a glycoside such as a galactoside. Herein, the glycoside moiety can be linked to the macrocycle through a self-immolative linker. An example of such compound capable of protecting the tetrapeptidic axle of the compound of the invention is described by e.g. Barat et al. 2015 (Chem Sci 6:2608-2613) and consists of a rotaxane or pseudo-rotaxane (as self-opening macrocycle) linked to a galactoside moiety (enabling removal by beta-galactosidase) via a self-immolative linker (in the case described being the nitro-benzyloxycarbonyl linker, eliminates itself after the deglycosylation reaction).

In a further embodiment, the capping group C and the tetrapeptidic moiety OP in the general compound structure C-OP-D are directly linked (or coupled or bound) to each other, or, alternatively are linked (or coupled or bound) indirectly via a linker or spacing group. A direct linkage between the capping group C and the tetrapeptidic moiety OP may be direct, e.g. via the N-terminal amino group of the tetrapeptidic moiety OP, or via a side chain of one of the amino acids of the tetrapeptidic moiety OP. Alternatively, said linkage may be indirect, e.g. by introducing a linker or spacer group between the tetrapeptidic moiety OP and the capping group C. Whatever the type of linkage (or coupling or bonding), direct or indirect, the linkage should: (1) not or not significantly disturb the functionality of the tetrapeptidic moiety, i.e., should not or not significantly disturb the proteolytic scissability of OP and (2) should retain the blood stability of the compound. Determination of the functionality of a linker or spacing group in the prodrug compound can be tested (e.g. stability in mammalian serum, selective toxicity to cancerous cells, etc.). Possible reasons for including a linker or spacing group between the capping group C and the tetrapeptidic moiety OP are the same as those listed hereinabove relating to the linker or spacing group between the tetrapeptidic moiety OP and the drug moiety D.

In a particular embodiment, the linker or spacer between the capping group C and the tetrapeptide moiety OP is not comprising a proteinaceous moiety such as an L-amino acid or a derivative of an L-amino acid. In a further embodiment, said linker or spacer is not comprising a D-amino acid or a derivative of a D-amino acid. In a further embodiment, said linker or spacer is not comprising a non-natural amino acid.

### Capping Group C

A protecting or capping moiety C, usually covalently linked to the N-terminal side of the oligopeptide, as present in the compounds of the current invention, adds to the solubility and/or stability of the prodrug compound (e.g. in mammalian blood or serum) and/or adds to the prevention of internalization of the prodrug compound into a cell such as a target cell. Such protecting or capping moieties include non-natural amino acids, β-alanyl or succinyl groups (e.g. WO96/05863, U.S. Pat. No. 5,962,216). Further stabilizing, protecting or capping moieties include diglycolic acid, maleic acid, pyroglutamic acid, glutaric acid, (e.g., WO00/33888), a carboxylic acid, adipic acid, phthalic acid, fumaric acid, naphthalene dicarboxylic acid, 1,8-naphtyldicarboxylic acid, aconitic acid, carboxycinnamic acid, triazole dicarboxylic acid, butane disulfonic acid, polyethylene glycol (PEG) or an analog thereof (e.g., WO01/95945), acetic acid, 1- or 2-naphthylcarboxylic acid, gluconic acid, 4-carboxyphenyl boronic acid, polyethylene glycolic acid, nipecotic acid, and isonipecotic acid (e.g., WO02/00263, WO02/100353), succinylated polyethylene glycol (e.g., WO01/91798). A new type of protecting or capping moiety was introduced in WO2008/120098, being a 1,2,3,4 cyclobutanetetracarboxylic acid. The protecting or capping moiety in WO02/07770 may be polyglutamic acid, carboxylated dextranes, carboxylated polyethylene glycol or a polymer based on hydroxyprolyl-methacrylamide or N-(2-hydroxyprolyl)methacryloylamide. Other capping groups include epsilon-maleimidocaproyl (Elsadek et al. 2010, Eur J Cancer 46:3434-3444), benzyloxycarbonyl (Dubowchik et al. 1998, Bioorg Med Chem Lett 8:3341-3346), and succinyl and phosphonoacetyl (e.g. WO 2014/102312).

In yet a further embodiment, (a) polyethylene glycol group(s) may be linked, coupled or bound to an amino acid, such as the N-terminal amino acid, of the tetrapeptidic moiety OP. Such pegylation may be introduced in order to increase the half-life of a compound C-OP-D in circulation after administration to a mammal and/or to increase solubility of a compound C-OP-D. Addition of (a) polyethylene glycol group(s)/pegylation could alternatively or additionally play the role of a capping agent.

### Drug Moiety D

The drug moiety D or therapeutic agent conjugated to the tetrapeptidic moiety OP of the invention may be useful for treatment of cancer (e.g. by exerting cytostatic, cytotoxic, anti-cancer or antiangiogenic activity; e.g. as adjuvant therapy, as part of a treatment regimen), inflammatory disease, or some other medical condition. The drug moiety D or therapeutic agent D may be any drug or therapeutic agent capable of entering a target cell (passively or by any uptake mechanism). Thus, the therapeutic agent may be selected from a number of classes of compounds including, alkylating agents, antiproliferative agents, tubulin binding agents, vinca alkaloids, enediynes, podophyllotoxins or podophyllotoxin derivatives, the pteridine family of drugs, taxanes, anthracyclines (and oxazolino anthracyclines, Rogalska et al. 2018n PLOS One 13:e0201296), dolastatins or their analogues (such as auristatins), topoisomerase inhibitors, platinum-coordination-complex chemotherapeutic agents, and maytansinoids.

More in particular, said drug moiety D or therapeutic agent may be one of the following compounds, or a derivative or analog thereof: doxorubicin and analogues [such as N-(5,5-diacetoxypent-1-yl)doxorubicin: Farquhar et al. 1998, J Med Chem 41:965-972; epirubicin (4′-epidoxorubicin), 4′-deoxydoxorubicin (esorubicin), 4′-iodo-4′-deoxydoxorubicin, and 4′-O-methyldoxorubicin: Arcamone et al. 1987, Cancer Treatment Rev 14:159-161 & Giuliani et al. 1980, Cancer Res 40:4682-4687; DOX-F-PYR (pyrrolidine analog of DOX), DOX-F-PIP (piperidine analog of DOX), DOX-F-MOR (morpholine analog of DOX), DOX-F-PAZ (N-methylpiperazine analog of DOX), DOX-F-HEX (hexamehtyleneimine analog of DOX), oxazolinodoxorubicin (3′deamino-3′-N, 4′-O-methylidenodoxorubicin, O-DOX): Denel-Bobrowska et al. 2017, Life Sci 178:1-8)], daunorubicin (or daunomycin) and analogues thereof [such as idarubicin (4′-demethoxydaunorubicin): Arcamone et al. 1987, Cancer Treatment Rev 14:159-161; 4′-epidaunorubicin; analogues with a simplified core structure bound to the monosaccharide daunosamine, acosamine, or 4-amino-2,3,6-trideoxy-L-threo-hexopyranose: see compounds 8-13 in Fan et al. 2007, J Organic Chem 72:2917-2928], amrubicin, vinblastine, vincristine, calicheamicin, etoposide, etoposide phosphate, CC-1065 (Boger et al. 1995, Bioorg Med Chem 3:611-621), duocarmycins (such as duocarmycin A and duocarmycin SA; Boger et al. 1995, Proc Natl Acad Sci USA 92:3642-3649), the duocarmycin derivative KW-2189 (Kobayashi et al. 1994, Cancer Res 54:2404-2410), methotrexate, methopterin, aminopterin, dichloromethotrexate, docetaxel, paclitaxel, epithiolone, combretastatin, combretastatin A4 phosphate, dolastatin 10, dolastatin 10 analogues (such as auristatins, e.g. auristatin E, auristatin-PHE, monomethyl auristatin D, monomethyl auristatin E, monomethyl auristatin F; see e.g. Maderna et al. 2014, J Med Chem 57:10527-10534), dolastatin 11, dolastatin 15, topotecan, exatecan, SN38, camptothecin, mitomycin C, porfiromycin, 5-fluorouracil, 6-mercaptopurine, fludarabine, tamoxifen, cytosine arabinoside, adenosine arabinoside, colchicine, halichondrin B, cisplatin, carboplatin, mitomycin C, bleomycin and analogues thereof (e.g. liblomycin, Takahashi et al. 1987, Cancer Treatment Rev 14:169-177), melphalan, chloroquine, cyclosporin A, and maytansine (and maytansinoids and analogues thereof such as analogues comprising a disulfide or thiol substituent: Widdison et al. 2006, J Med Chem 49:4392-4408; maytansin analogs DM1 and DM4). By derivative is intended a compound that results from reacting the named compound with another chemical moiety (different from the tetrapeptidic moiety linked directly or indirectly to the compound), and includes a pharmaceutically acceptable salt, acid, base, ester or ether of the named compound.

Other therapeutic agents or drugs include: vindesine, vinorelbine, 10-deacetyltaxol, 7-epi-taxol, baccatin III, 7-xylosyltaxol, isotaxel, ifosfamide, chloroaminophene, procarbazine, chlorambucil, thiophosphoramide, busulfan, dacarbazine (DTIC), geldanamycin, nitroso ureas, estramustine, BCNU, CCNU, fotemustine, streptonigrin, oxaliplatin, methotrexate, aminopterin, raltitrexed, gemcitabine, cladribine, clofarabine, pentostatin, hydroxyureas, irinotecan, topotecan, 9-dimethylaminomethyl-hydroxy-camptothecin hydrochloride, teniposide, amsacrine; mitoxantrone; L-canavanine, THP-adriamycin, idarubicin, rubidazone, pirarubicin, zorubicin, aclarubicin, epiadriamycin (4′epiadriamycin or epirubicin), mitoxantrone, bleomycins, actinomycins including actinomycin D, streptozotocin, calicheamycin; L-asparaginase; hormones; pure inhibitors of aromatase; androgens, proteasome inhibitors; farnesyl-transferase inhibitors (FTI); epothilones; discodermolide; fostriecin; inhibitors of tyrosine kinases such as STI 571 (imatinib mesylate); receptor tyrosine kinase inhibitors such as erlotinib, sorafenib, vandetanib, canertinib, PKI 166, gefitinib, sunitinib, lapatinib, EKB-569; Bcr-Abl kinase inhibitors such as dasatinib, nilotinib, imatinib; aurora kinase inhibitors such as VX-680, CYC116, PHA-739358, SU-6668, JNJ-7706621, MLN8054, AZD-1152, PHA-680632; CDK inhibitors such as flavopirodol, seliciclib, E7070, BMS-387032; MEK inhibitors such as PD184352, U-0126; mTOR inhibitors such as CCI-779 or AP23573; kinesin spindle inhibitors such as ispinesib or MK-0731; RAF/MEK inhibitors such as Sorafenib, CHIR-265, PLX-4032, CI-1040, PD0325901 or ARRY-142886; bryostatin; L-779450; LY333531; endostatins; the HSP 90 binding agent geldanamycin, macrocyclic polyethers such as halichondrin B, eribulin, or an analogue or derivative of any thereof.

The term “analogue” of a compound generally refers to a structural analogue or chemical analogue of that compound. Analogues include, but are not limited to isomers.

The term “derivative” of a compound refers to a compound that is structurally similar to and retains sufficient functional attributes of the original compound. The derivative may be structurally similar because one or more atoms are lacking, are substituted, are in different hydration/oxidation states, or because one or more atoms within the molecule are switched, such as, but not limited to, adding a hydroxyl group, replacing an oxygen atom with a sulfur atom, or replacing an amino group with a hydroxyl group, oxidizing a hydroxyl group to a carbonyl group, reducing a carbonyl group to a hydroxyl group, and reducing a carbon-to-carbon double bond to an alkyl group or oxidizing a carbon-to-carbon single bond to a double bond compared to the original compound. A derivative optionally has one or more, the same or different, substitutions. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

### Salts, Crystals, Co-Crystals, Polymorphs, Isomers

“Pharmaceutically acceptable”, as used herein, such as in the context of salts, crystals, co-crystals, polymorphs and isomers, means those salts of C-OP-D compounds of the invention that are safe and effective for the intended medical use. In addition, any of such salts, crystals, co-crystals, polymorphs and isomers that possess the desired biological activity.

Salts: Any of numerous compounds that result from replacement of part or all of an acidic or basic group present in a drug moiety D or compound C-OP-D of the invention. Suitable salts include, but are not limited to, aluminum, calcium, lithium, magnesium, potassium, sodium, zinc, and diethanolamine salts. For a review on pharmaceutically acceptable salts see, e.g., Berge et al. 1977 (J. Pharm. Sci. 66,1-19) or Handbook of Pharmaceutical Salts: Properties, Selection, and Use (P. H. Stahl, C. G. Wermuth (Eds.), August 2002), incorporated herein by reference. Per the current regulatory scheme, different salt forms of the same active moiety are considered different active pharmaceutical ingredients (APIs). (from: FDA draft guidance for industry “Regulatory Classification of Pharmaceutical Co-Crystals”; August 2016).

Polymorphs: Different crystalline forms of the same API. This may include solvation or hydration products (also known as pseudopolymorphs) and amorphous forms. Per the current regulatory scheme, different polymorphic forms are considered the same APIs. Lyophilization of an API often results in a dry powder comprising an amorphous form of the API.

Co-crystals: Crystalline materials composed of two or more different molecules within the same crystal lattice that are associated by nonionic and noncovalent bonds.

Co-crystals are crystalline materials composed of two or more different molecules, typically an API or drug and co-crystal formers (“coformers”), in the same crystal lattice. Pharmaceutical co-crystals have opened up opportunities for engineering solid-state forms beyond conventional solid-state forms of an API or drug, such as salts and polymorphs. Co-crystals are readily distinguished from salts because unlike salts, their components are in a neutral state and interact nonionically. In addition, co-crystals differ from polymorphs, which are defined as including only single-component crystalline forms that have different arrangements or conformations of the molecules in the crystal lattice, amorphous forms, and multicomponent phases such as solvate and hydrate forms. Instead co-crystals are more similar to solvates, in that both contain more than one component in the lattice. From a physical chemistry perspective, co-crystals can be viewed as a special case of solvates and hydrates, wherein the second component, the coformer, is nonvolatile. Therefore, co-crystals are classified as a special case of solvates in which the second component is nonvolatile.

Isomers: Stereoisomeric molecules, or stereoisomers, contain the same atoms linked together in the same sequence (same molecular formula), but having different three-dimensional organizations or configurations. Optical isomers, also sometimes referred to as enantiomers, are molecules which are non-superposable mirror images of each other. Depending on the optical activity, enantiomers are often described as left- or right-handed, and each member of the pair is referred to as enantiomorph (each enantiomorph being a molecule of one chirality). Mixtures of equal parts of two enantiomorphs are often referred to as racemic mixtures. Compounds comprising within the limits of detection only one enantiomorph are referred to as enantiopure compounds. Optical isomers can occur when molecules comprise one or more chiral centers. Geometric isomers usually refer to cis-trans isomers wherein rotation around a chemical bond is impossible. Cis-trans isomers often are found in molecules with double or triple bonds. Structural isomers contain the same atoms (same molecular formula), but linked together in a different sequence.

### Medicament

A further aspect of the invention relates to a compound C-OP-D (or salt, crystal, polymporph, isomer or amorphous form thereof, or co-crystal comprising it) as disclosed herein for use as a medicament or for use in the manufacture of a medicament. In one embodiment, the medicament is for use in e.g. the treatment of a cancer.

### Compositions

The invention relates to compositions comprising a salt of a compound C-OP-D, a crystal or co-crystal comprising a compound C-OP-D, a polymorph or amorphous form of a compound C-OP-D, or an isomer of a compound C-OP-D. In particular compositions comprising a pharmaceutically acceptable salt of a compound C-OP-D, a pharmaceutically acceptable crystal or co-crystal comprising a compound C-OP-D, a pharmaceutically acceptable polymorph of the compound C-OP-D, a pharmaceutically acceptable amporphous form of the compound C-OP-D, or a pharmaceutically acceptable isomer of a compound C-OP-D. Further in particular, such composition is a pharmaceutically acceptable composition and is further comprising at least one of a pharmaceutically acceptable solvent, diluent, or carrier.

A further aspect of the invention relates to compositions comprising a compound C-OP-D (or salt, crystal, polymporph, isomer or amorphous form thereof, or co-crystal comprising it) as disclosed herein.

Any of the above compositions can be used as medicament, or are for use in the manufacture of a medicament; such medicament is e.g. for use in the treatment of a cancer. In one embodiment, any of the above compositions is a pharmaceutically acceptable composition and is further comprising at least one of a pharmaceutically acceptable solvent, diluent, or carrier.

A composition of the invention thus can comprise besides the compound C-OP-D (or salt, crystal, polymporph, isomer or amorphous form thereof, or co-crystal comprising it) any one of a suitable solvent (capable of solubilizing the prodrug compound to the desired extent), diluent (capable of diluting concentrated prodrug compound to the desired extent) or carrier (any compound capable of absorbing, adhering or incorporating the prodrug compound, and of subsequently releasing at any rate the prodrug compound in the extracellular compartment of the subject's body). Said composition may alternatively comprise multiple (i.e. more than 1) prodrug compounds, or salt, crystal, polymporph, or amorphous form isomer thereof, or co-crystal comprising it, or any combination thereof (e.g. prodrug compound 1+its salt, prodrug compound 1+prodrug compound 2, prodrug compound 1+its salt+prodrug compound 2, etc.). In particular, said solvent, diluent or carrier is pharmaceutically acceptable, i.e., is acceptable to be administered to a subject to be treated with the composition of the invention. Aiding in formulating a pharmaceutically acceptable composition is e.g. any Pharmacopeia book. The composition may be formulated such that it is suitable for any way of administration including intra-cranial, intra-spinal, enteral, parenteral, intra-organ, intra-tumoral, intra-thecal, epidural etc. administration. The regimen by which the prodrug compound is administered may vary, e.g. depending on its pharmacokinetic characteristics, depending on the formulation, depending on the overall physical condition of a subject to be treated and e.g. depending on the judgment of the treating physician.

### Cancer

The compound C-OP-D (or salt, crystal, polymporph, isomer or amorphous form thereof, or co-crystal comprising it) of the invention, or a composition comprising it, is particularly suitable for treating a disease that is treatable by the released drug. Of particular interest is cancer or tumors such as solid tumors. “Cancer” includes e.g. breast cancers, soft tissue sarcoma, colorectal cancers, liver cancers, lung cancers such as small cell, non-small cell, bronchic cancers, prostate cancers, renal cancer, esophageal cancer, ovarian cancers, brain cancers, and pancreatic cancers, colon cancers, head and neck cancers, stomach cancers, bladder cancers, non-Hodgkin's lymphomas, leukaemias, neuroblastomas, glioblastomas, mesenchymal-like adenocarcinomas, basal-like adenocarcinomas, endometrioid adenocarcinomas, (metastatic) non-small cell lung carcinomas, (metastatic) melanomas, mucoepithelial pulmonary carcinomas, colon carcinomas, colon adenocarcinomas, prostate carcinomas, pancreatic ductal carcinomas.

### Treatment/Therapeutically Effective Amound

The subject to be treated with the compound C-OP-D (or salt, crystal, polymporph, isomer or amorphous form thereof, or co-crystal comprising it) of the invention can be any mammal in need of such treatment but is in particular a human. The treatment can result in regression of the disease [e.g. in terms of decreasing (primary) tumor volume or (primary) tumor mass and/or in terms of decreasing or inhibiting metastasis (e.g. number and/or growth of metastases), in decreased progression of the disease compared to expected disease progression, or in stabilization of the disease, i.e. neither regression nor progression of the disease. All these are favorable outcomes of the treatment. In particular, the effective amounts of said compound C-OP-D (or salt, crystal, polymporph, isomer or amorphous form thereof, or co-crystal comprising it), or of said composition is not causing severe leukopenia or cardiac toxicity/cardiotoxicity at therapeutic dosage. A possible definition of severe human leukopenia is WHO-criteria-defined grade 3-(1000-1900 leukocytes/mL) or grade 4-leukopenia (less than 1000 leukocytes/mL).

“Treatment”/“treating” refers to any rate of reduction, delay or retardation of the progress of the disease or disorder, or a single symptom thereof, compared to the progress or expected progress of the disease or disorder, or singe symptom thereof, when left untreated. This implies that a therapeutic modality on its own may not result in a complete or partial response (or may even not result in any response), but may, in particular when combined with other therapeutic modalities, contribute to a complete or partial response (e.g. by rendering the disease or disorder more sensitive to therapy). More desirable, the treatment results in no/zero progress of the disease or disorder, or singe symptom thereof (i.e. “inhibition” or “inhibition of progression”), or even in any rate of regression of the already developed disease or disorder or single symptom thereof. “Suppression/suppressing” can in this context be used as alternative for “treatment/treating”. Treatment/treating also refers to achieving a significant amelioration of one or more clinical symptoms associated with a disease or disorder, or of any single symptom thereof. Depending on the situation, the significant amelioration may be scored quantitatively or qualitatively. Qualitative criteria may e.g. be patient well-being. In the case of quantitative evaluation, the significant amelioration is typically a 10% or more, a 20% or more, a 25% or more, a 30% or more, a 40% or more, a 50% or more, a 60% or more, a 70% or more, a 75% or more, a 80% or more, a 95% or more, or a 100% improvement over the situation prior to treatment.

The time-frame over which the improvement is evaluated will depend on the type of criteria/disease observed and can be determined by the person skilled in the art.

A “therapeutically effective amount” refers to an amount of a therapeutic agent to treat or prevent a disease, disorder, or unwanted condition in a subject. The term “effective amount” refers to the dosing regimen of the agent or composition comprising the agent (e.g. medicament or pharmaceutical composition). The effective amount will generally depend on and/or will need adjustment to the mode of contacting or administration. The effective amount of the agent or composition comprising the agent is the amount required to obtain the desired clinical outcome or therapeutic effect without causing significant or unnecessary toxic effects (often expressed as maximum tolerable dose, MTD).

To obtain or maintain the effective amount, the agent or composition comprising the agent may be administered as a single dose or in multiple doses (see explanation on single administrations), such as to obtain or maintain the effective amount over the desired time span/treatment duration. The effective amount may further vary depending on the severity of the condition that needs to be treated; this may depend on the overall health and physical condition of the mammal or patient and usually the treating doctor's or physician's assessment will be required to establish what is the effective amount. The effective amount may further be obtained by a combination of different types of contacting or administration.

The aspects and embodiments described above in general may comprise the administration of one or more therapeutic compounds to a subject in need thereof, i.e., in need of treatment. In general a (therapeutically) effective amount of (a) therapeutic compound(s) is administered to the subject in need thereof in order to obtain the described clinical response(s). “Administering” means any mode of contacting that results in interaction between an agent (e.g. a therapeutic compound) or composition comprising the agent (such as a medicament or pharmaceutical composition) and an object (e.g. cell, tissue, organ, body lumen) with which said agent or composition is contacted.

Administering can e.g. be parenteral administration (intravenous, intramuscular, subcutaneous), intrathecal administration, intracerebral administration, epidural administration, intracardial administration, intraosseous administration, intraperitoneal administration, (mini)pump-controlled administration, administration in the vicinity of a cancer or tumor, administration via a catheter or a peripherally inserted central catheter or percutaneous indwelling central catheter, and includes e.g. bolus administration. The interaction between the agent or composition and the object can occur starting immediately or nearly immediately with the administration of the agent or composition, can occur over an extended time period (starting immediately or nearly immediately with the administration of the agent or composition), or can be delayed relative to the time of administration of the agent or composition. More specifically the “contacting” results in delivering an effective amount of the agent or composition comprising the agent to the object.

A single administration of a pharmacologic compound in general leads to a transient effect due to its gradual removal from the cell, organ and/or body and is reflected in the pharmacokinetic/-dynamic behavior of the compound. Depending on the desired level of therapeutic agent, two or more (multiple) administrations of the pharmacologic compound may thus be required.

### Combination/Combination Therapy

“Combination” or “combination in any way” or “combination in any appropriate way” as referred to herein is meant to refer to any sequence of administration of two (or more) therapeutic modalities, i.e. the administration of the two (or more) therapeutic modalities can occur concurrently or separated from each other for any amount of time; and/or “combination”, “combination in any way” or “combination in any appropriate way” as referred to herein can refer to the combined or separate formulation of the two (or more) therapeutic modalities, i.e. the two (or more) therapeutic modalities can be individually provided in separate vials or (other suitable) containers, or can be provided combined in the same vial or (other suitable) container. When combined in the same vial or (other suitable) container, the two (or more) therapeutic modalities can each be provided in the same vial/container chamber of a single-chamber vial/container or in the same vial/container chamber of a multi-chamber vial/container; or can each be provided in a separate vial/container chamber of a multi-chamber vial/container. The therapeutic modalities of the current invention are a compound of the formula C-OP-D (or salt, crystal, polymporph, isomer or amorphous form thereof, or co-crystal comprising it) and an immune checkpoint inhibitor.

Combinations of a compound C-OP-D (or salt, crystal, polymporph, isomer or amorphous form thereof, or co-crystal comprising it) as disclosed herein with a chemotherapeutic agent and/or with one or more alkylating antineoplastic agent(s) and/or one or more anti-metabolite(s) and/or one or more anti-microtubule agent(s) and/or one or more topoisomerase inhibitor(s) and/or one or more cytotoxic antibiotic(s) and/or one or more (biological) anticancer agent(s) (such as antibodies) and/or with one or more immunotherapeutic agents are one aspect of the invention.

Inclusion of a compound C-OP-D (or salt, crystal, polymporph, isomer or amorphous form thereof, or co-crystal comprising it) according to the present invention in combination therapies is also envisaged. In particular for treatment of a tumor or cancer, this can be in a combined modality chemotherapy, i.e. the use of the anticancer compound C-OP-D (or salt, crystal, polymporph, isomer or amorphous form thereof, or co-crystal comprising it) with other cancer treatments, such as radiation therapy (whether by direct irradiation or via administering an isotope-labeled antibody or antibody fragment) or surgery. This can also be in combination chemotherapy, i.e. treating a patient with a number of different drugs wherein the drugs preferably differ in their mechanism of action and in their side effects. In such combination chemotherapy the different drugs can be administered simultaneously (but not necessarily combined in a single composition) or separated in any order one relative to another. An advantage of combination chemotherapy is the minimization of the chance of the development of resistance to any one agent. A further advantage may be that the individual drugs can each be used at a lower dose, thereby reducing overall toxicity.

A compound C-OP-D (or salt, crystal, polymporph, isomer or amorphous form thereof, or co-crystal comprising it) according to the invention, or a composition comprising such compound C-OP-D (or salt, crystal, polymporph, isomer or amorphous form thereof, or co-crystal comprising it), can thus be used (in a method) for manufacture of a medicament; such as for manufacture of a medicament for treatment of a disease (e.g. cancer), as monotherapy, or as part of a combination chemotherapy treatment or a combined modality chemotherapy treatment.

A compound C-OP-D (or salt, crystal, polymporph, isomer or amorphous form thereof, or co-crystal comprising it) according to the invention, or a composition comprising such compound C-OP-D (or salt, crystal, polymporph, isomer or amorphous form thereof, or co-crystal comprising it), can thus be used (in a method) for treatment of a disease (e.g. cancer), as monotherapy, or as part of a combination chemotherapy treatment or a combined modality chemotherapy treatment. In a method of treatment of a disease, the compound C-OP-D (or salt, crystal, polymporph, isomer or amorphous form thereof, or co-crystal comprising it), or a composition comprising it, is administered to a subject in need, therewith treating the disease. In particular, a therapeutically effective dose or therapeutically effective dose regimen of a compound C-OP-D (or salt, crystal, polymporph, isomer or amorphous form thereof, or co-crystal comprising it), or of a composition comprising it, is administered to the subject in need, therewith treating the disease. A subject in need in general is a subject, such as a mammal, having, suffering from, or diagnosed to have the disease.

More in general in relation to combination chemotherapy, an anticancer compound C-OP-D (or salt, crystal, polymporph, isomer or amorphous form thereof, or co-crystal comprising it) according to the invention can be combined with one or more alkylating antineoplastic agent(s) and/or one or more anti-metabolite(s) and/or one or more anti-microtubule agent(s) and/or one or more topoisomerase inhibitor(s) and/or one or more cytotoxic antibiotic(s) and/or one or more (biological) anticancer agent(s) (such as antibodies). When applicable, one or more of these can in one embodiment be included in a prodrug compound(s) (or a salt thereof) according to the present invention. In another embodiment, the prodrug compound(s) according to the present invention are not combined with a free drug D when D is present in said prodrug compound(s). Alternatively, the prodrug compound(s) according to the present invention can be combined with one or more alkylating antineoplastic agent(s) different from D and/or one or more anti-metabolite(s) different from D and/or one or more anti-microtubule agent(s) different from D and/or one or more topoisomerase inhibitor(s) different from D and/or one or more cytotoxic antibiotic(s) different from D, wherein D is part of the prodrug compound C-OP-D as disclosed herein.

Immunotherapy is a promising new area of cancer therapeutics and several immunotherapies are being evaluated preclinically as well as in clinical trials and have demonstrated promising activity (Callahan et al. 2013, J Leukoc Biol 94:41-53; Page et al. 2014, Annu Rev Med 65:185-202). However, not all the patients are sensitive to immune checkpoint blockade and sometimes PD-1 or PD-L1 blocking antibodies accelerate tumor progression. To this purpose, combinatorial cancer treatments that include chemotherapies can achieve higher rates of disease control by impinging on distinct elements of tumor biology to obtain synergistic antitumor effects. It is now accepted that certain chemotherapies can increase tumor immunity by inducing immunogenic cell death and by promoting escape in cancer immunoediting. Any compound C-OP-D (or salt, crystal, polymporph, isomer or amorphous form thereof, or co-crystal comprising it) according to the invention can be combined with immunotherapeutic agents such as, but not limited to, immune checkpoints antagonists. Immune checkpoints antagonists or inhibitors as referred to herein include the cell surface protein cytotoxic T lymphocyte antigen-4 (CTLA-4), programmed cell death protein-1 (PD-1) and their respective ligands. CTLA-4 binds to its co-receptor B7-1 (CD80) or B7-2 (CD86); PD-1 binds to its ligands PD-L1 (B7-H10) and PD-L2 (B7-DC). Other immune checkpoint inhibitors include the adenosine A2A receptor (A2AR), B7-H3 (or CD276), B7-H4 (or VTCN1), BTLA (or CD272), IDO (indoleamine 2,3-dioxygenase), KIR (killer-cell immunoglobulin-like receptor), LAG3 (lymphocyte activation gene-3), NOX2 (nicotinamide adenine dinucleotide phosphate (NADPH) oxidase isoform 2), TIM3 (T-cell immunoglobulin domain and mucin domain 3), VISTA (V-domain Ig suppressor of T cell activation), SIGLEC7 (sialic acid-binding immunoglobulin-type lectin 7, or CD328) and SIGLEC9 (sialic acid-binding immunoglobulin-type lectin 9, or CD329). In a particular embodiment the immune checkpoint antagonists or inhibitors are selected for inclusion in a combination or combination therapy (as outlined above).

In particular, any compound C-OP-D (or salt, crystal, polymporph, isomer or amorphous form thereof, or co-crystal comprising it) according to the invention capable of inducing immunogenic cell death can be combined with an immunotherapeutic agent. Drug moieties D known to induce immunogenic cell death include bleomycin, bortezomib, cyclophosphamide, doxorubicin, epirubicin, idarubicin, mafosfamide, mitoxantrone, oxaliplatin, and patupilone (Bezu et al. 2015, Front Immunol 6:187).

The drug doxorubicin (also known under trade names such as Adriamycin or Rubex) is commonly used to treat multiple types of cancers such as some leukemias and Hodgkin's lymphoma, as well as cancers of the bladder, breast, stomach, lung, ovaries, thyroid, soft tissue sarcoma, multiple myeloma, and others. Doxorubicin is further used in different combination therapies. Doxorubicin-containing therapies include AC or CA (Adriamycin, cyclophosphamide), TAC (Taxotere, AC), ABVD (Adriamycin, bleomycin, vinblastine, dacarbazine), BEACOPP (bleomycin, etoposide, Adriamycin (doxorubicin), cyclophosphamide, Oncovin (vincristine), procarbazine, prednisone), CHOP (cyclophosphamide, Adriamycin, vincristine, prednisolone), FAC or CAF (5-fluorouracil, Adriamycin, cyclophosphamide), MVAC (methothrexate, vincristine, adriamycin, cisplatin), CAV (cyclophosphamide, doxorubicin, vincristine) and CAVE (CAV, etoposide), CVAD (cyclophosphamide, vincristine, adriamycin, dexamethasone), DT-PACE (dexamethasone, thalidomide, cisplatin or platinol, adriamycin, cyclophosphamide, etoposide), m-BACOD (methothrexate, bleomycin, adriamycin, cyclophosphamide, vincristine, dexamethasone), MACOP-B (methothrexate, leucovorin, adriamycin, cyclophosphamide, vincristine, prednisone, bleomycin), Pro-MACE-MOPP (methothrexate, adriamycin, cyclophosphamide, etoposide, mechlorethamine, vincristine, procarbazine, prednisone), ProMACE-CytaBOM (prednisone, doxorubicin, cyclophosphamide, etoposide, cytarabine, bleomycin, vincristine, methothrexate, leucovorin), Stanford V (doxorubicin, mechlorethamine, bleomycin, vinblastine, vincristine, etoposide, prednisone), DD-4A (vincristine, actinomycin, doxorubicin), VAD (vincristine, doxorubicin, dexamethasone), Regimen I (vincristine, doxorubicin, etoposide, cyclophosphamide) and VAPEC-B (vincristine, doxorubicin, prednisone, etoposide, cyclophosphamide, bleomycin). Besides the doxorubicin-comprising combination chemotherapies there is a plethora of other combination chemotherapies such as BEP (Bleomycin, etoposide, platinum agent (cisplatin (Platinol))), CAPOX or XELOX (capecitabine, oxaliplatin), CBV (cyclophosphamide, carmustine, etoposide), FOLFIRI (fluorouracil, leucovorin, irinotecan), FOLFIRINOX (fluorouracil, leucovorin, irinotecan, oxaliplatin), FOLFOX (fluorouracil, leucovorin, oxaliplatin), EC (epirubicin, cyclophosphamide), ICE (ifosfamide, carboplatin, etoposide (VP-16)) and IFL (irinotecan, leucovorin, fluorouracil). Combination of doxorubicin with sirolimus (rapamycin) has been disclosed by Wendel et al. 2004 (Nature 428, 332-337) in treatment of Akt-positive lymphomas in mice. In any of these combination therapies, doxorubicin could be substituted by a compound C-OP-D (or salt, crystal, polymporph or isomer thereof, or co-crystal comprising it) as disclosed herein and wherein D is doxorubicin.

One can further also envisage combination therapies including an anticancer compound C-OP-D (or salt, crystal, polymporph, isomer or amorphous form thereof, or co-crystal comprising it) according to the invention (whether alone or already part of a combination chemotherapy or of a combined modality therapy) and compounds other than cytostatics. Such other compounds include any compound approved for treating cancer or being developed for treating cancer. In particular, such other compounds include monoclonal antibodies such as alemtuzumab (chronic lymphocytic leukemia), bevacizumab (colorectal cancer), cetuximab (colorectal cancer, head and neck cancer), denosumab (solid tumor's bony metastases), gemtuzumab (acute myelogenous leukemia), ipilimumab (melanoma), ofatumumab (chronic lymphocytic leukemia), panitumumab (colorectal cancer), rituximab (Non-Hodgkin lymphoma), tositumomab (Non-Hodgkin lymphoma) and trastuzumab (breast cancer). Other antibodies include for instance abagovomab (ovarian cancer), adecatumumab (prostate and breast cancer), afutuzumab (lymphoma), amatuximab, apolizumab (hematological cancers), blinatumomab, cixutumumab (solid tumors), dacetuzumab (hematologic cancers), elotuzumab (multiple myeloma), farletuzumab (ovarian cancer), intetumumab (solid tumors), matuzumab (colorectal, lung and stomach cancer), onartuzumab, parsatuzumab, pritumumab (brain cancer), tremelimumab, ublituximab, veltuzumab (non-Hodgkin's lymphoma), votumumab (colorectal tumors), zatuximab and anti-placental growth factor antibodies such as described in WO 2006/099698. Examples of such combination therapies include for instance CHOP-R (CHOP (see above)+rituximab), ICE-R (ICE (see above)+rituximab), R-FCM (rituximab, fludarabine, cyclophosphamide, mitoxantrone) and TCH (Paclitaxel (Taxol), carboplatin, trastuzumab).

Examples of alkylating antineoplastic agents include nitrogen mustards (for example mechlorethamine, cyclophosphamide, melphalan, chlorambucil, ifosfamide and busulfan), nitrosoureas (for example N-Nitroso-N-methylurea (MNU), carmustine (BCNU), lomustine (CCNU), semustine (MeCCNU), fotemustine and streptozotocin), tetrazines (for example dacarbazine, mitozolomide and temozolomide), aziridines (for example thiotepa, mytomycin and diaziquone (AZQ)), cisplatins and derivatives (for example cisplatin, carboplatin and oxaliplatin), and non-classical alkylating agents (for example procarbazine and hexamethylmelamine)

Subtypes of the anti-metabolites include the anti-folates (for example methotrexate and pemetrexed), fluoropyrimidines (for example fluorouracil, capecitabine and tegafur/uracil), deoxynucleoside analogues (for example cytarabine, gemcitabine, decitabine, Vidaza, fludarabine, nelarabine, cladribine, clofarabine and pentostatin) and thiopurines (for example thioguanine and mercaptopurine).

Anti-microtubule agents include the vinca alkaloid subtypes (for example vincristine, vinblastine, vinorelbine, vindesine and vinflunine) and taxane subtypes (for example paclitaxel and docetaxel). Other anti-microtubule agents include podophyllotoxin.

Topoisomerase inhibitors include topoisomerase I inhibitors (for example irinotecan, topotecan, camptothecin, exatecan, and SN-38 which is the active metabolite of irinotecan) and topoisomerase II inhibitors (for example etoposide, doxorubicin, mitoxantrone, teniposide, novobiocin, merbarone, and aclarubicin).

Cytotoxic drugs further include anthracyclines (doxorubicin, daunorubicin, epirubicin, idarubicin, pirarubicin, aclarubicin and mitoxantrone) and other drugs including actinomycin, bleomycin, plicamycin and mitomycin.

Other anti-cancer drugs include CDK4/6 inhibitors such as palbociclib (PD-0332991), ribociclib, or abemaciclib.

Other anti-cancer drugs include inhibitors of poly(ADP-ribose) polymerases (PARP), such as niraparib, olaparib, rucaparib, talazoparib, rucaparib, veliparib, CEP-9722, BSI-201, INO-1001, or PJ34.

Any anticancer compound C-OP-D (or salt, crystal, polymporph, isomer or amorphous form thereof, or co-crystal comprising it) according to the invention can (whether alone or already part of a combination chemotherapy or of a combined modality therapy) further be included in an antibody-directed enzyme prodrug therapy (ADEPT), which includes the application of cancer-associated monoclonal antibodies, which are linked, to a drug-activating enzyme. Subsequent systemic administration of a non-toxic agent results in its conversion to a toxic drug, and resulting in a cytotoxic effect which can be targeted at malignant cells (Bagshawe et al. (1995) Tumor Targeting 1, 17-29.)

Further, any anticancer compound C-OP-D (or salt, crystal, polymporph, isomer or amorphous form thereof, or co-crystal comprising it) according to the invention can (whether alone or already part of a combination chemotherapy or of a combined modality therapy) be combined with one or more agent(s) capable of reversing (multi)drug resistance ((M)DR reverser(s) or (M)DR reversing agent(s)) that can occur during chemotherapy. Such agents include for example loperamide (Zhou et al. 2011, Cancer Invest 30, 119-125). Another such combination includes loading the prodrug compound in nanoparticles such as iron oxide nanoparticles (Kievit et al. 2011, J Control Release 152, 76-83) or liposomes. Examples of drugs loaded into liposomes include doxorubicin (doxorubicin HCL liposomes, also known under the trade names Doxil, Caelyx or Myocet), daunorubicin (known under the trade name DaunoXome) and paclitaxel (Garcion et al. 2006, Mol Cancer Ther 5, 1710-1722).

A compound C-OP-D (or salt, crystal, polymporph, isomer or amorphous form thereof, or co-crystal comprising it) according to the invention, or a composition comprising such compound C-OP-D (or salt, crystal, polymporph, isomer or amorphous form thereof, or co-crystal comprising it), can thus be used for manufacturing a medicament; such as a medicament for treating a disease (e.g. cancer), as monotherapy, or as part of a combination chemotherapy treatment or a combined modality chemotherapy treatment. A compound C-OP-D (or salt, crystal, polymporph, isomer or amorphous form thereof, or co-crystal comprising it) according to the invention, or a composition comprising such compound C-OP-D (or salt, crystal, polymporph, isomer or amorphous form thereof, or co-crystal comprising it), can thus be used (in a method) for treatment of a disease (e.g. cancer), as monotherapy, or as part of a combination chemotherapy treatment or a combined modality chemotherapy treatment. Any of such treatments can further be combined with a treatment including a drug resistance reverting agent.

In an embodiment thereto, a compound C-OP-D (or salt, crystal, polymporph, isomer, or amorphous form thereof, or co-crystal comprising it) according to the invention, or a composition comprising such compound C-OP-D (or salt, crystal, polymporph, isomer or amorphous form thereof, or co-crystal comprising it) is applied in a combination chemotherapy treatment or a combined modality chemotherapy treatment and the drug moiety D is effective or therapeutically effective as cytotoxic, cytostatic, or anti-cancer drug in a combination chemotherapy treatment or a combined modality chemotherapy treatment.

### Synthesis or Production of C-OP-D

In a further aspect, the invention relates to methods for synthesizing or producing a compound C-OP-D.

In general, a method for producing a compound C-OP-D, is a method comprising the steps of: linking the drug D, the tetrapeptidic moiety OP, and the capping group C; wherein the linking of D, OP and C is resulting in the compound C-OP-D, and wherein the linking between drug D and tetrapeptidic moiety OP and/or between capping group C and tetrapeptidic moiety OP is direct or via a linker or spacing group.

In particular embodiments, such method for synthesizing or producing a compound C-OP-D, is a method wherein:
the drug D is linked to the capped oligopeptide moiety complex C-OP, resulting in the compound C-OP-D; or
wherein the drug D is linked to the tetrapeptidic moiety OP and the capping group C is linked to the tetrapeptidic moiety-drug complex OP-D, resulting in the compound C-OP-D; or
wherein the drug D is linked to an intermediate of the tetrapeptidic moiety OP, the intermediate of the tetrapeptidic moiety is extended, and the capping group C is linked to the tetrapeptidic moiety-drug complex OP-D, resulting in the compound C-OP-D; or
wherein the drug D is linked to an intermediate of the tetrapeptidic moiety OP, the intermediate of the tetrapeptidic moiety is extended with the remainder of the tetrapeptidic moiety to which the capping group C is already attached, resulting in the compound C-OP-D; or
wherein the drug D is linked to an intermediate of the tetrapeptidic moiety OP, the intermediate of the tetrapeptidic moiety is extended in one or more steps of which one step is extension with an amino acid to which the capping group C is already attached, resulting in the compound C-OP-D; or
wherein in any of the above the drug D is coupled to the complex C-OP, to the tetrapeptidic moiety OP, or to an intermediate of the tetrapeptidic moiety OP, via a linker or spacing group; or
wherein in any of the above the drug D itself coupled to a linker or spacing group is coupled, via the linker or spacing group, to the complex C-OP, to the tetrapeptidic moiety OP, or to the intermediate of the tetrapeptidic moiety OP; or
wherein in any of the above a linker or spacing group itself coupled to the complex C-OP, to the tetrapeptidic moiety OP, or to the intermediate of the tetrapeptidic moiety OP, is coupled to the drug D, wherein the linker or spacing group is structurally in between the complex C-OP, the tetrapeptidic moiety OP, or the intermediate of the tetrapeptidic moiety OP on the one hand, and the drug D on the other hand; or
wherein the capping group C is linked, directly or indirectly, to the tetrapeptidic moiety OP and the complex C-OP is linked, directly or indirectly, to the drug D, resulting in the compound C-OP-D; or
wherein the capping group C is linked, directly or indirectly, to an intermediate of the tetrapeptidic moiety OP, the intermediate of the tetrapeptidic moiety is extended, and the drug D is linked, directly or indirectly, to the complex C-OP, resulting in the compound C-OP-D.

In the above-described methods for producing a compound C-OP-D, in one of the steps:
the capping group C may be introduced on the tetrapeptidic moiety OP during the synthesis of OP; or
the linker or spacing group may be introduced on the tetrapeptidic moiety OP during the synthesis of OP, or is introduced on the drug D (prior to linking to tetrapeptidic moiety OP).

Any of the above-described methods for producing a compound C-OP-D may further comprise a step of purifying the compound C-OP-D.

Any of the above-described methods for producing a compound C-OP-D may further comprise a step of forming a salt, crystal, co-crystal, polymorph or amorphous form of the compound C-OP-D.

As described above, said linking of the tetrapeptidic moiety OP with the drug D and/or capping group C may be direct, or indirect via a linker or spacing group, such as a self-immolating or self-eliminating spacer. The purification strategy of the prodrug compound will obviously depend on the nature of the drug and/or of the capping group and/or of the tetrapeptidic moiety OP. A skilled person will be able to design a suitable purification strategy for any possible compound according to the invention, choosing from a plethora of purification techniques that are available.

### Kits

The invention further relates to kits comprising a container comprising compound C-OP-D (or salt, crystal, polymporph, isomer or amorphous form thereof, or co-crystal comprising it) according to the invention or comprising a composition comprising such prodrug compound or salt thereof. Such kit may further comprise, in the same container (holding a compound according to the invention) or in one or more separate containers, one or more further anticancer drugs, such as an antibody or fragment thereof (e.g. as described above). Alternatively, or in addition, such kit may further comprise, in the same container (holding a compound according to the invention) or in one or more separate containers, one or more drug resistance reversing agents. Other optional components of such kit include one or more diagnostic agents capable of prognosing, predicting or determining the success of a therapy comprising a compound according to the invention; use instructions; one or more containers with sterile pharmaceutically acceptable carriers, excipients or diluents [such as for producing or formulating a (pharmaceutically acceptable) composition of the invention]; one or more containers with agents for ADEPT therapy; etc.

### Other Definitions

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Terms or definitions described hereinabove and hereunder are provided solely to aid in the understanding of the invention. Unless specifically defined herein, all terms used herein have the same meaning as they would to one skilled in the art of the present invention. In relation to molecular biology, practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. None of the definitions provided herein should not be construed to have a scope less than understood by a person of ordinary skill in the art.

The term “defined by SEQ ID NO:X” as used herein refers to a biological sequence consisting of the sequence of amino acids or nucleotides given in the SEQ ID NO:X. For instance, an antigen defined in/by SEQ ID NO:X consists of the amino acid sequence given in SEQ ID NO:X. A further example is an amino acid sequence comprising SEQ ID NO:X, which refers to an amino acid sequence longer than the amino acid sequence given in SEQ ID NO:X but entirely comprising the amino acid sequence given in SEQ ID NO:X (wherein the amino acid sequence given in SEQ ID NO:X can be located N-terminally or C-terminally in the longer amino acid sequence, or can be embedded in the longer amino acid sequence), or to an amino acid sequence consisting of the amino acid sequence given in SEQ ID NO:X. All references hereinabove and hereinafter cited are incorporated in their entirety by their reference.

### EXAMPLES

### Abbreviations

Dox: Doxorubicin; MMAE: monomethyl auristatin E (monomethylvaline-valine-dolaisoleuine-dolaproine-norephedrine); TNBC: triple negative breast cancer; CrC: colorectal cancer; GBM: glioblastoma multiforme; PrC: prostate cancer; PaC: pancreatic cancer; OvC: ovarian cancer; NSCLC: non-small cell lung cancer; hiPSCs: human induced pluripotent stem cells; PhAc: phosphonoacetyl; ALGP (SEQ ID NO:3): alanyl-leucyl-glycyl-prolyl (Ala Leu Gly Pro); ALLP (SEQ ID NO:1): alanyl-leucyl-leucyl-prolyl (Ala Leu Leu Pro); ALKP (SEQ ID NO:2): alanyl-leucyl-lysyl-prolyl (Ala Leu Lys Pro); PABC: p-aminobenzylcarbamate; alCso: absolute IC₅₀ (concentration required to kill 50% of the cells). DMF: N,N-Dimethylformamide; DIC: N,N′-Diisopropylcarbodiimide; HOBt: 1-Hydroxybenzotriazole; DIEA: N,N-Diisopropylethylamine; TMSBr: Trimethylsilyl bromide.

### Example 1. Chemical Synthesis of Auristatin- and Doxorubicin-Comprising Prodrug Compounds and of Intermediates

The chemical synthesis of auristatin- and doxorubicin-comprising compounds is described hereafter. Synthesis of prodrugs comprising ALGP (SEQ ID NO:3) as tetrapeptidic moiety OP, without capping group or with succinyl or phosphonacetyl as capping group C, and doxorubicin as drug D has been described in Example 1 of WO 2014/102312. The skilled person will understand that synthesis of these compounds is enabling chemical synthesis of similar compounds with other tetrapeptidic moieties (in particular the tetrapeptidic moieties ALLP (SEQ ID NO:1), APKP (SEQ ID NO:2)). Synthesis of prodrugs comprising ALGP (SEQ ID NO:3) as tetrapeptidic moiety OP, phosphonacetyl as capping group C, and with drug D either being maytansine, geldanamycin, paclitaxel, docetaxel, camptothecin, vinblastine, vincristine, methotrexate, aminopterin, and amrubicin are described in Example 16 of WO 2014/102312.

When present, the linker or spacing group PABC (p-aminobenzyloxycarbamate; alternatively p-aminobenzyloxycarbonyl) is introduced between the tetrapeptidic moiety OP and the drug D; PABC is removed via a spontaneous 1,6 benzyl elimination mechanism after proteolytic removal of OP. The ortho version of PABC could likewise be used, and is removed via a spontaneous 1,4-elimination. The introduction of the PABC linker is described hereinafter in case of the drug D being auristatin. It can likewise be introduced in the tetrapeptidic prodrug wherein the drug D is doxorubicin (see e.g. Elsadek et al. 2010, ACS Med Chem Lett 1:234-238).

### Compound 1: PhAc-ALGP-PABC-MMAE [Compound 2]

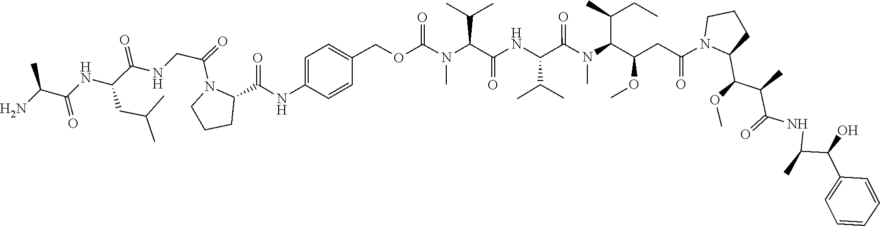 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@@H]2CCCN2C(=O)CNC(=O)[C@H](CC(C)C)NC(=O)[C@H](C)N)cc1)C(C)C]

[Compound 1] MMA-E (also referred to herein interchangeably as MMAE or auristatin) MMA-E was purchased from commercial supplier.

### Preparation of Intermediate 1

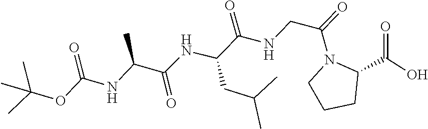 [SMILES: CC(C)C[C@H](NC(=O)[C@H](C)NC(=O)OC(C)(C)C)C(=O)NCC(=O)N1CCC[C@H]1C(=O)O]

Standard Fmoc peptide synthesis as previously described was used to prepare Boc-Ala-Leu-Gly-Pro (5 or 20 mmol scale).

### Preparation of Intermediate 2

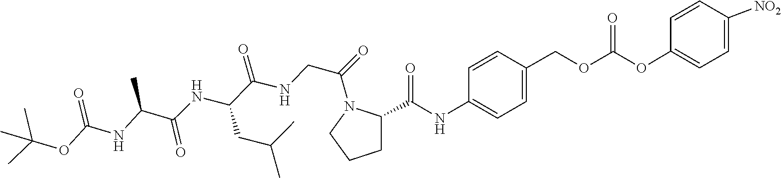 [SMILES: CC(C)C[C@H](NC(=O)[C@H](C)NC(=O)OC(C)(C)C)C(=O)NCC(=O)N1CCC[C@H]1C(=O)Nc1ccc(COC(=O)Oc2ccc([N+](=O)[O-])cc2)cc1]

To a solution of Intermediate 1 (1.5 g, 3.29 mmol) in DCM (10 mL) and MeOH (5 ml) was added EEDQ (1.63 g, 6.57 mmol, 2 eq) and 4-aminobenzyl alcohol (485.56 mg, 3.94 mmol, 1.2 eq). The mixture was stirred at 15° C. for 16 h. The reaction mixture was concentrated under reduced pressure and the residue was purified by prep-HPLC to afford the benzyl alcohol compound (0.6 g, 1.07 mmol, 32.5% yield).

To a solution of the previous compound (0.6 g, 1.07 mmol) in DMF (5 mL) was added Bis(4-nitrophenyl) carbonate (6 eq) and DIEA (6 eq). The solution was stirred at 15° C. for 16 h. The reaction mixture was subsequently concentrated under reduced pressure and the residue was purified by prep-HPLC to afford Intermediate 8 as a white solid (0.75 g, 96.4% yield).

Compound 1 was obtained from intermediate 2 and MMA-E (750 mg, 56% yield, white solid), followed by the Boc group deprotection (490 mg, 71% yield, white solid) by using similar procedure than for Compound 1.
Appearance: white solid
Purity by HPLC: >96%
Retention Time: 12.154 min
Mass spectrometry: 1205.6 [M+H]⁺

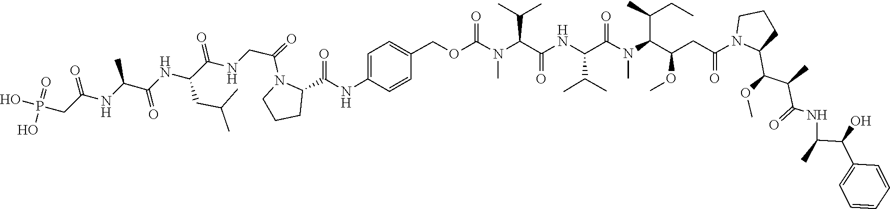 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@@H]2CCCN2C(=O)CNC(=O)[C@H](CC(C)C)NC(=O)[C@H](C)NC(=O)CP(=O)(O)O)cc1)C(C)C]

### [Compound 2] PhAc-ALGP-PABC-MMAE

Compound 2 was obtained by coupling compound 1 and 2-phosphonoacetic acid using HATU/DIEA in DMF. Compound 2 was isolated in 19% yield after purification by prep-HPLC.
Appearance: white solid
Purity by HPLC: >96%
Retention Time: 14.950
Mass spectrometry: 1327.5 [M+H]⁺

### Compound 3: PhAc-ALLP-Doxorubicin

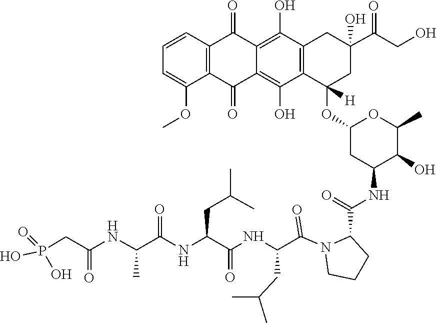 [SMILES: COc1cccc2c1C(=O)c1c(O)c3c(c(O)c1C2=O)C[C@@](O)(C(=O)CO)C[C@@H]3O[C@H]1C[C@H](NC(=O)[C@@H]2CCCN2C(=O)[C@H](CC(C)C)NC(=O)[C@H](CC(C)C)NC(=O)[C@H](C)NC(=O)CP(=O)(O)O)[C@H](O)[C@H](C)O1]

### [Compound 3] PhAc-ALLP-Doxorubicin

Peptide diethyl-PhAc-ALLP (PhAc: phosphonoacetyl moiety) was synthesized by standard solid phase Fmoc peptide (CTC resin, HBTU coupling (Pro, Leu, Leu, Ala) or DIC coupling (2-(diethoxyphosphoryl)acetic acid)).

The proline residue is then activated by N-hydroxysuccinimide (HOSu) in dichlomethane to obtain the diethyl phosphonyl acetyl ester (EtO)₂P(O)—CH₂—C(O)NH-ALAP-OSu. The phosponyl acid moiety is then deprotected with 0.5M TMsBr in DCM overnight. (HO)₂P(O)—CH₂—C(O)NH-ALLP-OSu is obtained by precipitation with cold methyl-tert-butyl ether. After drying, doxorubicin hydrochloride is coupled to the activated peptide in DCM in presence of DIEA. After 3h of reaction, the mixture is concentrated under reduced pressure and the residue is subsequently purified by preparative HPLC to give the title compound as a red powder (purity: 95%).

### Compound 4: PhAc-ALLP-PABC-MMAE

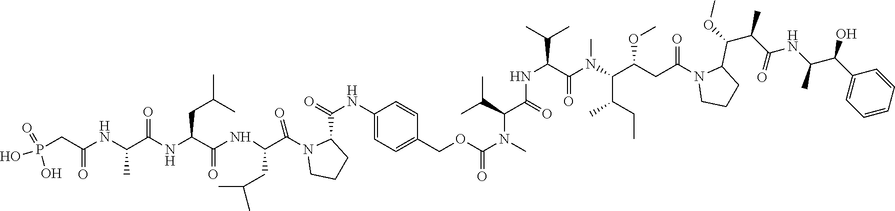 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@@H]2CCCN2C(=O)[C@H](CC(C)C)NC(=O)[C@H](CC(C)C)NC(=O)[C@H](C)NC(=O)CP(=O)(O)O)cc1)C(C)C]

### [Compound 4] PhAc-ALLP-PABC-MMAE

The peptide diethyl-PhAc-ALLP-OH was prepared by standard solid phase synthesis as described for Compound 3.

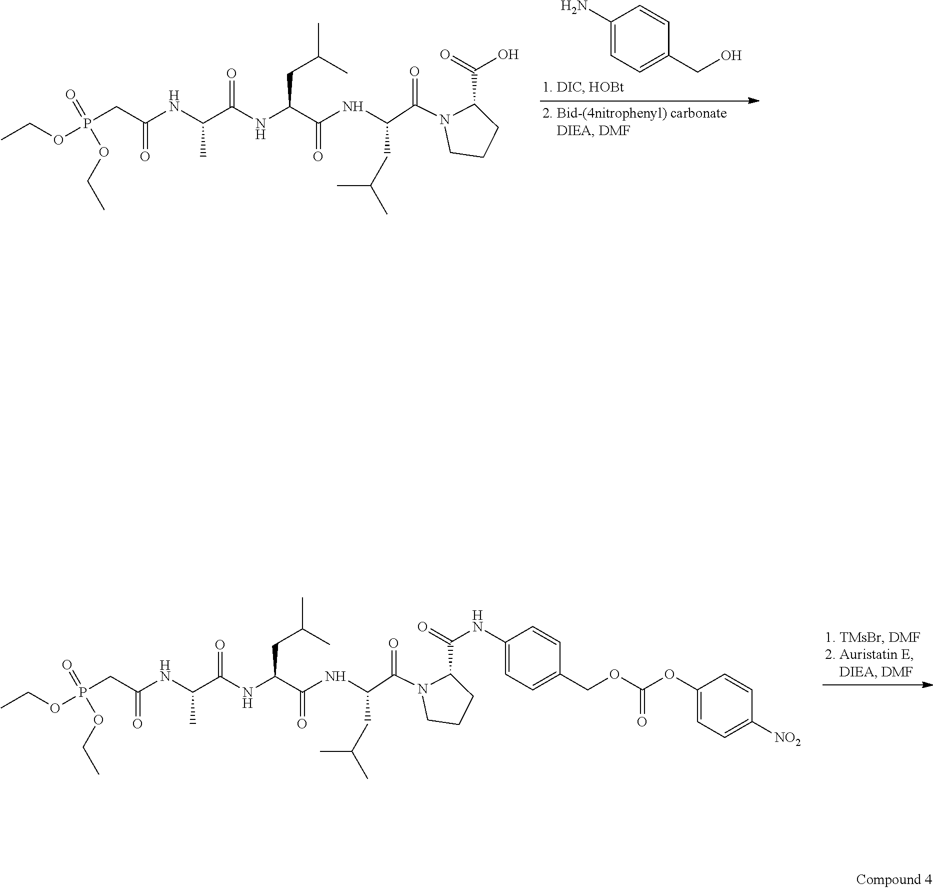 [SMILES: CCOP(=O)(CC(=O)N[C@@H](C)C(=O)N[C@@H](CC(C)C)C(=O)N[C@@H](CC(C)C)C(=O)N1CCC[C@H]1C(=O)O)OCC ; Nc1ccc(CO)cc1 ; CCOP(=O)(CC(=O)N[C@@H](C)C(=O)N[C@@H](CC(C)C)C(=O)N[C@@H](CC(C)C)C(=O)N1CCC[C@H]1C(=O)Nc1ccc(COC(=O)Oc2ccc([N+](=O)[O-])cc2)cc1)OCC]

4-Aminobenzyl alcohol is subsequently coupled to the previous peptide using DIC and HOBt in DMF. Bis(4-nitrophenyl) carbonate and DIEA were then added to a solution of the previous intermediate in DMF. The diethyl-phosphonioacetyl ester is deprotected by TMsBR in DMF and finally Auristatin E is condensed with the previous carbonate derivative in DMF with DIEA. The final compound is purified by preparative HPLC and eventually converted as sodium salt.
Appearance: white solid
Purity by HPLC: 98.8%
Retention Time: 12.154 min
Mass spectrometry: 1384 [M+H]⁺, 692.7 [M+2H]²⁺

### Example 2. Evaluation of ALLP- and APKP-Tetrapeptide-Comprising Prodrug Compounds of doxorubicin

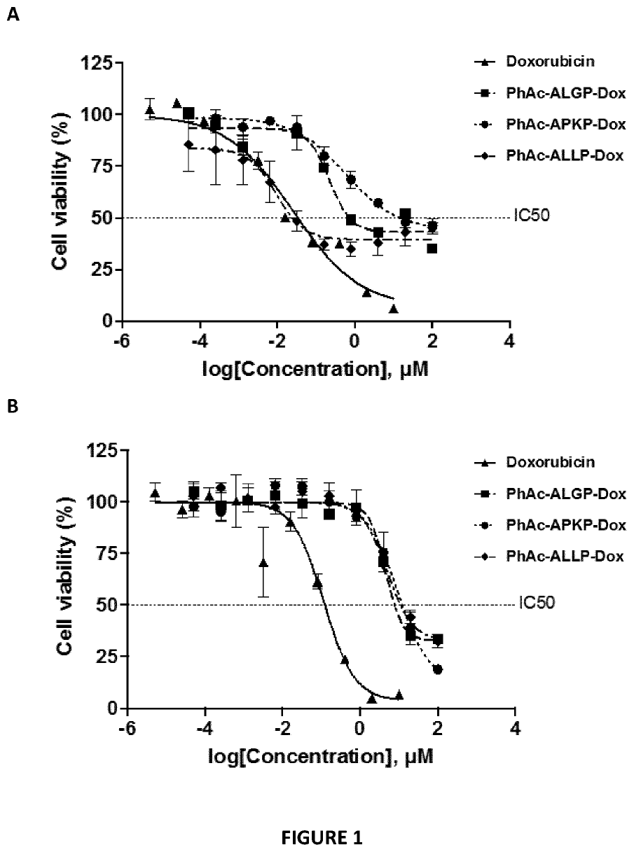

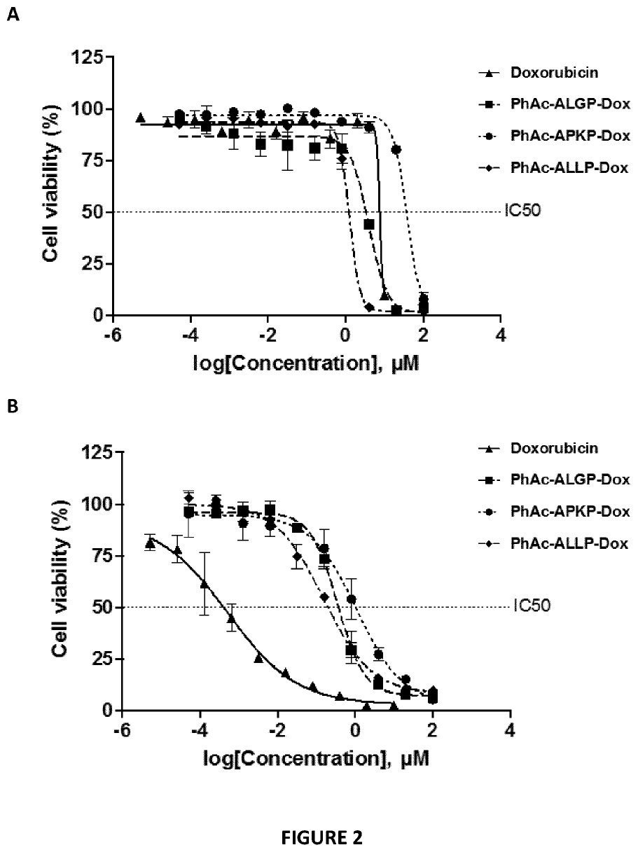

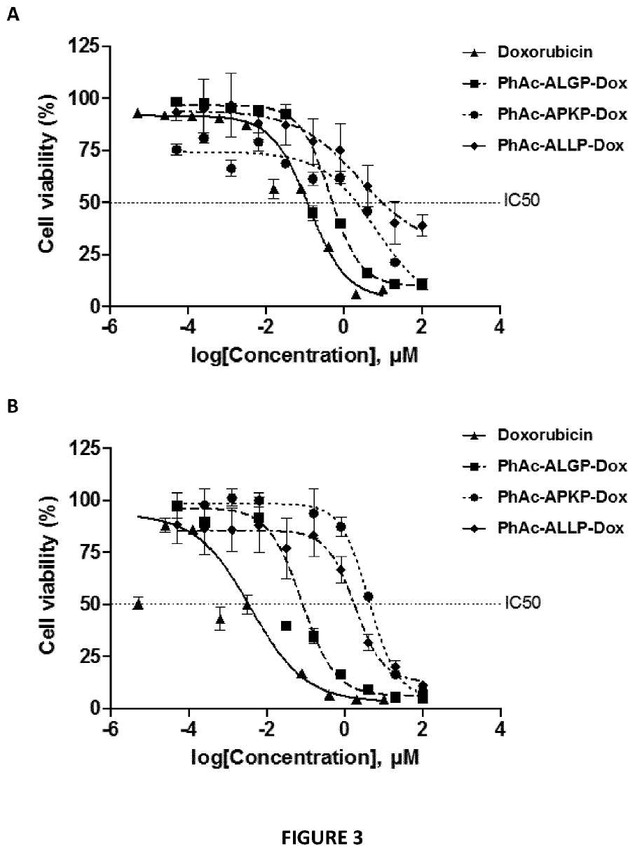

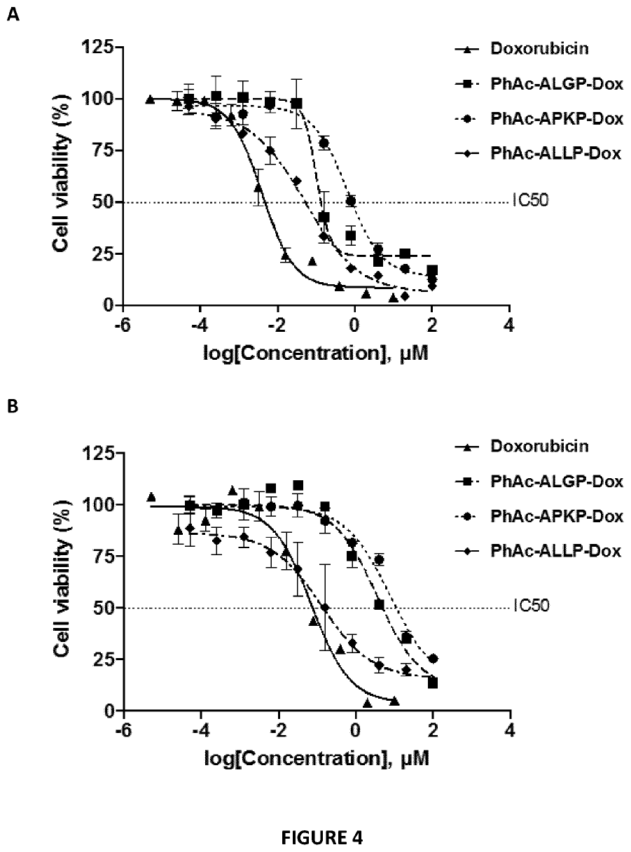

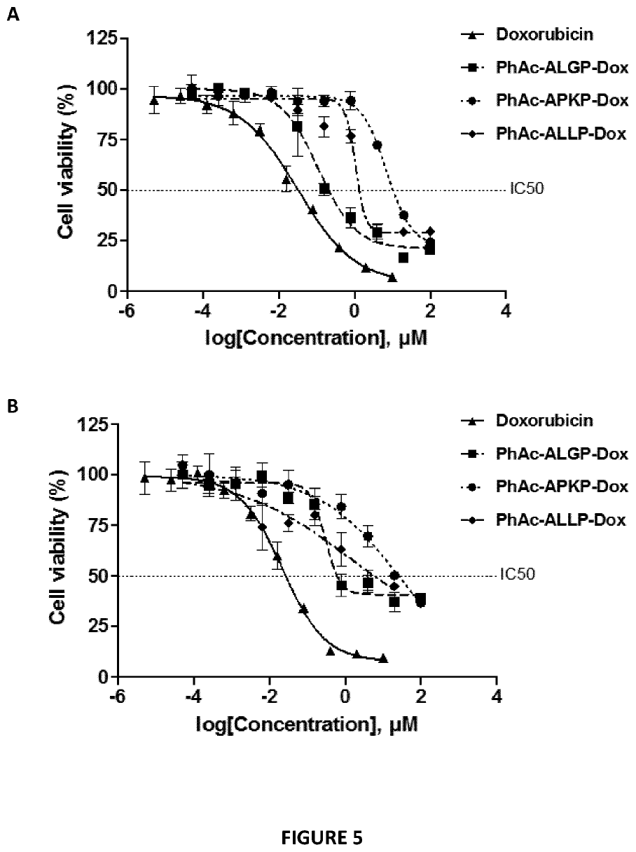

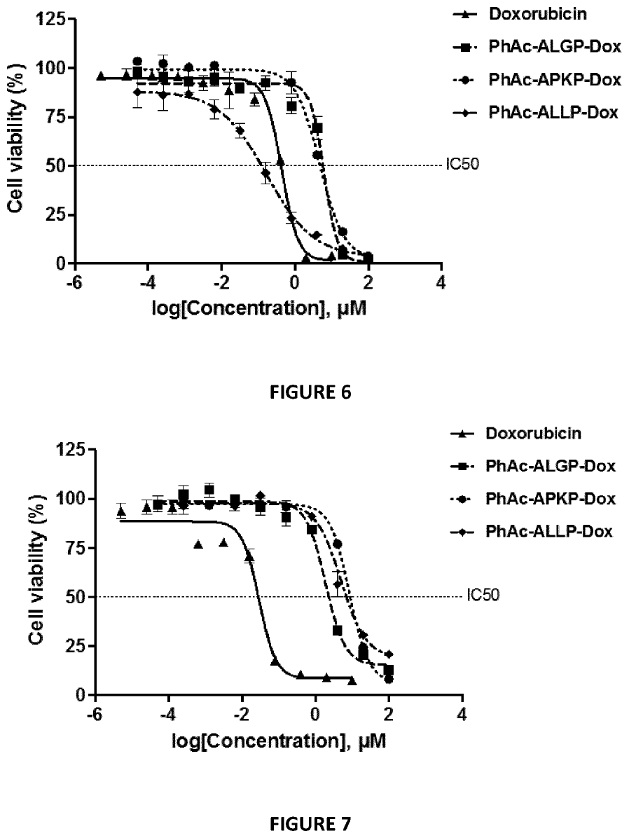

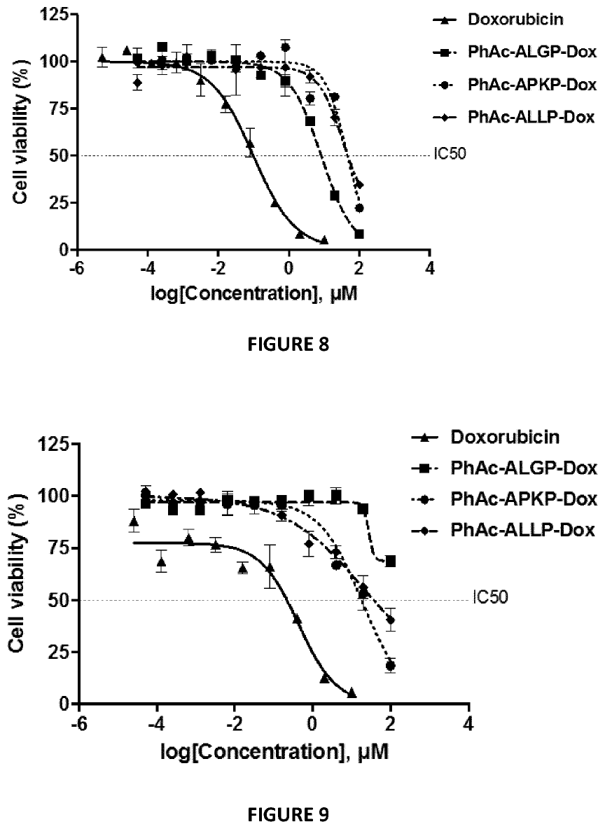

Prodrugs of doxorubicin comprising the tetrapeptide ALLP or APKP were synthetized and analyzed for their in vitro potency in a variety of cancer indications (FIGS. 1-8 and Table 1), this taking along the molecule PhAc-ALGP-Dox as described in WO2014/102312. The potency of the parent drug molecule (free doxorubicin) was on average 4.5 to 608 times higher when compared to the prodrug-version, depending on the indication. Both PhAc-ALLP-Dox and PhAc-APKP-Dox were able to effectively target cancer cells within the micromolar range. Where PhAc-APKP-Dox revealed similar micromolar potency against most cancer cell lines, PhAc-ALLP-Dox exerted more indication specific cytotoxicity, with the most favorable equipotency compared to the parent free drug doxorubicin in melanoma, ovarian cancer, colorectal cancer and glioblastoma (GBM).

TABLE 1
In vitro potency ALLP- and APKP-tetrapeptide-comprising prodrug compounds of doxorubicin
in a variety of cancer indications. Cells were seeded in a 96-well plate according to their optimal
cell densities (5.000-15.000 cells/well). Absolute IC₅₀ values (μM) based on cell viability
assessment after 72 hrs continuous drug exposure (WST-1). Sigmoidal-4PL non-linear fittings of
a 10-point serial titration, ranging from 100 μM to 2.048 nM, were used to extrapolate the
aIC₅₀. Values represent mean of triplicate measurements.

| | | PaC | CrC | | GBM | | TNBC | |
|---|---|---|---|---|---|---|---|---|
| | Normal | MIA | LS | HCT- | A- | U-87 | MDA- | MDA- |
| Compound | HME-1 | PaCa-2 | 174T | 116 | 172 | MG | MB-231 | MB-468 |
| Doxorubicin | 0.04 | 0.10 | 0.01 | 0.12 | 3.36 | 0.07 | 0.12 | <0.01 |
| PhAc-ALGP-Dox | >100 | 8.32 | 0.24 | 0.46 | 7.52 | 0.47 | 0.51 | 0.08 |
| PhAc-APKP-Dox | 18.07 | 45.71 | 14.72 | 12.29 | 36.94 | 12.40 | 2.13 | 4.49 |
| PhAc-ALLP-Dox | 36.90 | 46.77 | 0.02 | 11.06 | 1.21 | 0.29 | 9.57 | 1.76 |

| | Normal | PrC | OvC | | Melanoma | NSCLC | |
|---|---|---|---|---|---|---|---|
| Compound | HME-1 | DU145 | A2780 | A2780 Cp | A2058 | NCI-1299 | NCI-H292 |
| Doxorubicin | 0.04 | 0.03 | <0.01 | 0.08 | 0.41 | 0.03 | 0.03 |
| PhAc-ALGP-Dox | >100 | 2.34 | 0.13 | 4.90 | 5.79 | 0.21 | 0.62 |
| PhAc-APKP-Dox | 18.07 | 8.93 | 0.86 | 11.17 | 4.90 | 10.52 | 25.89 |
| PhAc-ALLP-Dox | 36.90 | 7.02 | 0.05 | 0.14 | 0.12 | 1.35 | 5.86 |

The maximal efficacy was assessed in the available cancer cell lines (Table 2). Consistent with its high potency, PhAc-ALLP-Dox was exceedingly effective with most pronounced cytotoxicity in GBM, melanoma, OvC, and CrC (Table 2).

TABLE 2
Maximal efficacy of ALLP- and APKP-tetrapeptide-comprising prodrug compounds
of doxorubicin in a variety of cancer indications. Cells were seeded in
a 96-well plate according to their optimal cell densities (5.000-15.000
cells/well). Cell viability was assessed after 72 hrs continuous drug exposure
(WST-1). Sigmoidal-4PL non-linear fittings of a 10-point serial titration,
ranging from 100 μM to 2.048 nM. Maximal efficacy was defined as the
cytotoxicity (%) at 100 μM. Experiment was run in triplicate.

| | | PaC | CrC | | GBM | | TNBC | |
|---|---|---|---|---|---|---|---|---|
| | Normal | MIA | LS | HCT- | A- | U-87 | MDA- | MDA- |
| Compound | HME-1 | PaCa-2 | 174T | 116 | 172 | MG | MB-231 | MB-468 |
| Doxorubicin | 96.50 | 94.44 | 93.74 | 93.45 | 89.90 | 71.78 | 91.49 | 95.52 |
| PhAc-ALGP-Dox | 31.17 | 91.53 | 64.94 | 66.44 | 96.32 | 69.54 | 88.97 | 94.90 |
| PhAc-APKP-Dox | 81.55 | 77.65 | 53.72 | 81.05 | 91.93 | 88.38 | 89.12 | 91.83 |
| PhAc-ALLP-Dox | 59.52 | 65.41 | 54.86 | 67.49 | 98.08 | 94.35 | 71.20 | 87.18 |

| | Normal | PrC | OvC | | Melanoma | NSCLC | |
|---|---|---|---|---|---|---|---|
| Compound | HME-1 | DU145 | A2780 | A2780 Cp | A2058 | NCI-1299 | NCI-H292 |
| Doxorubicin | 96.50 | 92.47 | 96.00 | 94.85 | 96.98 | 92.87 | 90.37 |
| PhAc-ALGP-Dox | 31.17 | 87.15 | 82.68 | 86.43 | 97.79 | 79.14 | 60.64 |
| PhAc-APKP-Dox | 81.55 | 91.93 | 87.34 | 74.61 | 96.27 | 75.52 | 63.26 |
| PhAc-ALLP-Dox | 59.52 | 79.25 | 90.59 | 85.27 | 97.56 | 70.15 | 55.15 |

While significant potency is one essential element of a prodrug, selectivity towards cancer cells over normal non-cancerous cells is another essential and possibly even more important element of any prodrug. Calculation of the absolute IC₅₀ (i.e. the concentration required to kill 50% of the cells) in normal cells over cancer cells, offers a well-accepted way of anticipating the selectivity of these compounds. As described by Basida et al. 2009 (Anticancer Res 29:2993-2996), compounds with a selectivity index greater than 2, potentially exert an increased therapeutic window. Therefore, normal Human Mammary Epithelium (HME-1) cells were similarly exposed to parent free drug and to the prodrugs comprising the tetrapeptide ALLP or APKP (FIG. 9 and Table 3). PhAc-ALLP-Dox and PhAc-APKP-Dox required significantly higher concentrations to reach equipotent toxicity in normal cells when compared to the parent free drug doxorubicin. On average, selectivity indices (SI) of the prodrugs consistently exceeded those of free doxorubicin by a factor of at least 2, with the exception of pancreatic cancer (MIA PaCa-2). In comparison to the benchmark compound PhAc-ALGP-Dox, PhAc-ALLP-Dox exerted relevant superiority in GBM, melanoma and certain subtypes of colorectal cancer (i.e. Dukes type B adenocarcinoma).

Taken together, these results highlight the potential of PhAc-ALLP-Dox and PhAc-APKP-Dox and for further in vivo validation.

TABLE 3
Selectivity indices of ALLP- and APKP-tetrapeptide-comprising prodrug compounds of doxorubicin.
Absolute IC₅₀ values (μM) based on cell viability assessment after 72 hrs continuous drug
exposure (WST-1). Sigmoidal-4PL non-linear fittings of a 10-point serial titration, ranging from 100
μM to 2.048 nM, were used to extrapolate the aIC₅₀. Experiment was run in triplicate (n =
3). The selectivity index was defined as the ratio of the concentration of drug required to kill
50% of the cells in normal cells, divided by the concentration required to exert the same efficacy
in tumor cells. Cells were seeded in a 96-well plate according to their optimal cell densities
(5.000-15.000 cells/well). Therefore, a SI below 1 is considered not selective towards tumor
cells, while the higher the index, the better the selectivity. When SI is greater than 2, these
compounds could potentially have a more beneficial therapeutic window (1).

| SI = ^{HME-1}IC₅₀/ | | PaC | CrC | | GBM | | TNBC | |
|---|---|---|---|---|---|---|---|---|
| ^{cancer}IC₅₀ | Normal | MIA | LS | HCT- | A- | U-87 | MDA- | MDA- |
| Sequence | HME-1 | PaCa-2 | 174T | 116 | 172 | MG | MB-231 | MB-468 |
| Doxorubicin | 1.00 | 0.44 | 4.47 | 0.36 | 0.01 | 0.59 | 0.38 | 12.64 |
| PhAc-ALGP-Dox* | 1.00 | >12.07 | >411.15 | >217.39 | >13.29 | >211.16 | >195.37 | >1182.61 |
| PhAc-APKP-Dox | 1.00 | 0.40 | 1.23 | 1.47 | 0.49 | 1.46 | 8.49 | 4.02 |
| PhAc-ALLP-Dox | 1.00 | 0.79 | 1514.9 | 3.34 | 30.54 | 128.53 | 3.85 | 20.94 |

SI = ^{HME-1}IC₅₀/

| | | | | | |
|---|---|---|---|---|---|
| ^{cancer}IC₅₀ | Normal | PrC | OvC | Melanoma | NSCLC |

| Sequence | HME-1 | DU145 | A2780 | A2780 Cp | A2058 | NCI-1299 | NCI-H292 |
|---|---|---|---|---|---|---|---|
| Doxorubicin | 1.00 | 1.55 | 9.15 | 0.57 | 0.11 | 1.33 | 1.62 |
| PhAc-ALGP-Dox* | 1.00 | >42.73 | >742.82 | >20.42 | >17.26 | >473.90 | >162.04 |
| PhAc-APKP-Dox | 1.00 | 2.02 | 21.13 | 1.62 | 3.69 | 1.72 | 0.70 |
| PhAc-ALLP-Dox | 1.00 | 5.25 | 701.33 | 257.22 | 298.19 | 27.39 | 6.30 |

*For PhAc-ALGP-Dox, IC₅₀ in normal cells exceeded the highest concentration tested (100 μM). As such, the selectivity is underestimated and exceeding the value reported.

### Example 3. Evaluation of ALLP-Tetrapeptide-Comprising Prodrug Compounds of Doxorubicin and auristatin

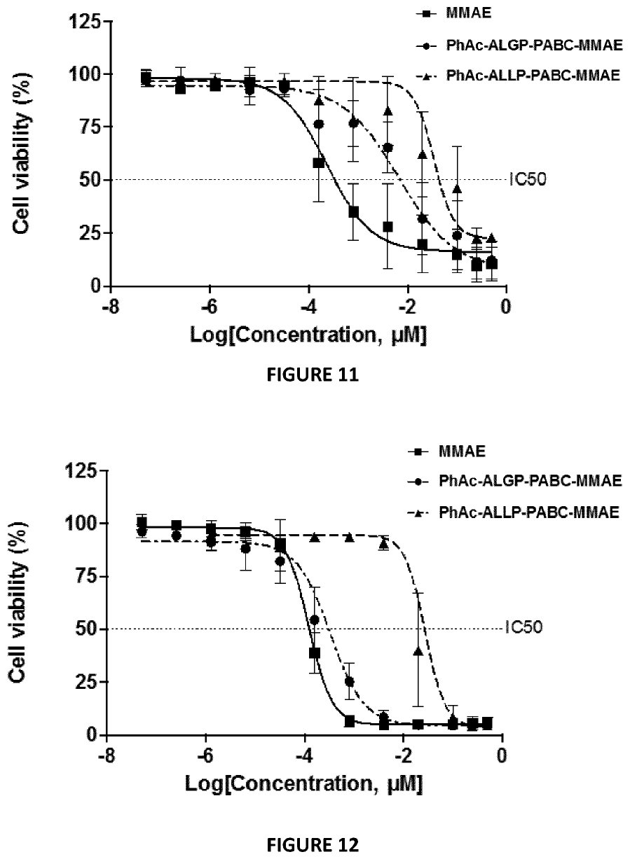

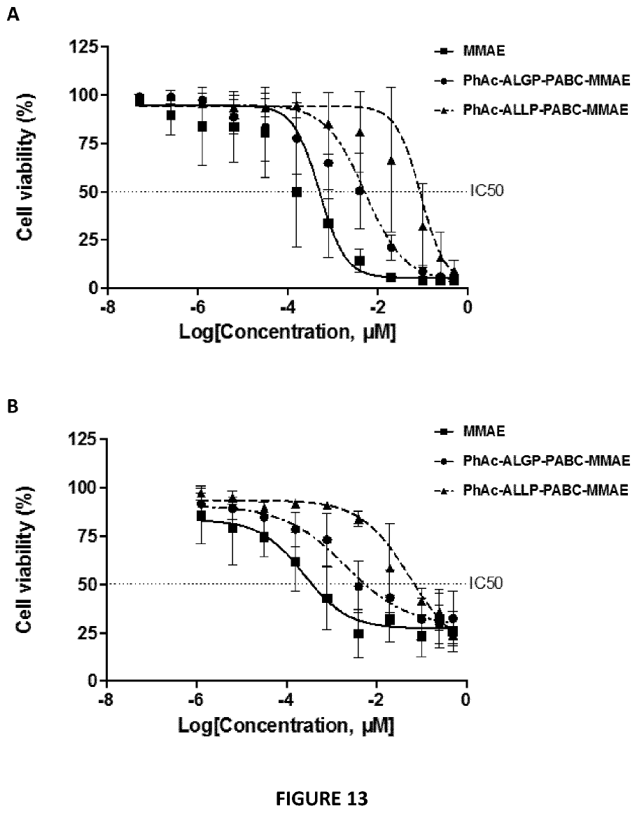

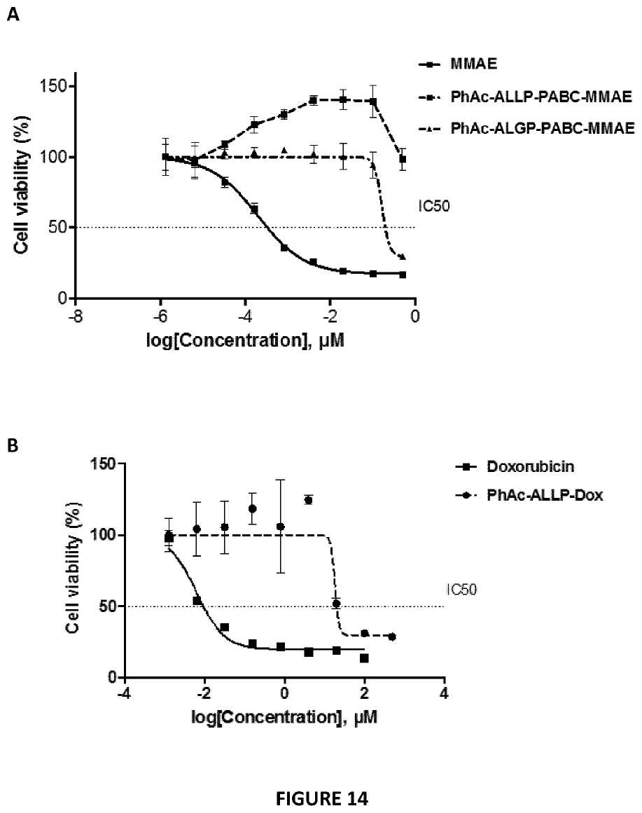

Monomethyl Auristatin E (MMAE) was selected as alternative drug to be incorporated in ALLP-tetrapeptide based prodrugs. MMAE is a synthetic microtubule interfering agent derived from dolastatins, having nanomolar potency but characterized by a lack of in vivo therapeutic window. Toxicity and selectivity of the resulting compound PhAc-ALLP-PABC-MMAE was assessed in the cancer indications TNBC, GMB and melanoma (FIGS. 11-13 and Tables 4-6). When compared to doxorubicin-comprising prodrugs, the reduction in potency of PhAc-ALLP-PABC-MMAE compared to free MMAE was more pronounced, but more importantly within low nanomolar range, highlighting the potential of MMAE-based prodrugs. Toxicity of PhAc-ALLP-PABC-MMAE compared to MMAE was not significantly different on mammary epithelial (HME-1) cells. However, when selectivity was determined on Human umbilical vein endothelial cells (HUVECs), an increased safety was apparent for all three indications (SI=3.8 for TNBC, 2.2 for GBM and 6.2 for melanoma) (FIG. 10 and Tables 4-6). Within these indications, potency and maximal efficacy were highest in A2058 melanoma cells (0.03 μM and 95.2% respectively) (Table 5). When considering PhAc-ALLP-PABC-MMAE as a therapeutic candidate for GBM, non-cancer associated astrocytes could be considered as a relevant cell type to assess the cancer-specific selectivity. Therefore, hiPSC-derived differentiated astrocytes (Type I) were used to calculate the selectivity index (Table 6). Importantly, PhAc-ALLP-PABC-MMAE completely lacked efficacy towards this cell type, indicating there is no activation of this prodrug outside the tumor micro-environment. As such, selectivity index exceeded the artificial threshold of 10× and maximal cytotoxicity was a mere 2%. Since ALLP appeared to exert indication-specific selectivity for GBM, also PhAc-ALLP-Dox cytotoxicity was evaluated towards iAstro's. Also for this prodrug compound, the excellent selectivity was confirmed (SI=14.9±0.8) (FIG. 14 and Tables 4-6), highlighting the potential of ALLP in GBM, regardless of the toxic moiety present in the ALLP-tetrapeptide based prodrug.

TABLE 4
In vitro potency of ALLP-based prodrugs. Either normal cells (HME-1, HUVEC
or iAstro's) or cancer cells (A-172, U-87 MG, A2058 and MDA-MB-231)
were seeded in a 96-well plate according to their optimal cell densities (5.000-
10.000 cells/well). Absolute IC₅₀ values (μM) based on cell viability
assessment after 72 hrs continuous drug exposure (WST-1). Sigmoidal-4PL non-linear
fittings of a 10-point serial titration, starting from 500 nM (MMAE and PhAc-
ALLP-PABC-MMAE) or 100 μM (PhAc-ALLP-Dox), were used to extrapolate the
aIC₅₀. Experiment was run in triplicate.

| | | | | GBM | | | TNBC |
|---|---|---|---|---|---|---|---|
| Absolute IC₅₀ (μM) | Normal | | | A- | U-87 | Melanoma | MDA- |
| Compound | HME-1 | HUVEC | iAstro | 172 | MG | A2058 | MB-231 |
| MMAE | 0.0001 | 0.0009 | 0.0003 | 0.0005 | 0.0004 | 0.0001 | 0.0003 |
| PhAc-ALLP-PABC- | 0.01 | 0.17 | >0.50 | 0.09 | 0.07 | 0.03 | 0.04 |
| MMAE | | | | | | | |
| PhAc-ALLP-Dox | 36.90 | 41.59 | 20.32 | 1.29 | 1.44 | 0.12 | 9.57 |

TABLE 5
Maximal efficacy of new of ALLP-based prodrugs. Either normal cells (HME-1, HUVEC
or iAstro's) or cancer cells (A-172, U-87 MG, A2058 and MDA-MB-231) were seeded
in a 96-well plate according to their optimal cell densities (5.000-10.000 cells/well).
Cell viability was assessed after 72 hrs continuous drug exposure (WST-1). Sigmoidal-
4PL non-linear fittings of a 10-point serial titration, starting from 100 μM (CBR-
014) of 500 nM (MMAE and CBR-073). Maximal efficacy was defined as the cytotoxicity
at 100 μM. Experiment was run in triplicate.

| | | | | GBM | | | TNBC |
|---|---|---|---|---|---|---|---|
| Maximal efficacy | Normal | | | A- | U-87 | Melanoma | MDA- |
| Sequence | HME-1 | HUVEC | iAstro | 172 | MG | A2058 | MB-231 |
| MMAE | 83.8 | 87.4 | 83.0 | 94.6 | 72.3 | 95.2 | 83.8 |
| PhAc-ALLP-PABC-MMAE | 99.5 | 98.1 | 1.7 | 97.4 | 86.6 | 95.2 | 77.6 |
| PhAc-ALLP-Dox | 59.5 | 53.1 | 68.5 | 80.9 | 97.1 | 97.6 | 71.2 |

TABLE 6
Selectivity indices of ALLP-based prodrugs. Absolute IC₅₀
values (μM) based on cell viability assessment after 72 hrs
continuous drug exposure (WST-1). Sigmoidal-4PL non-linear
fittings of a 10-point serial titration were used to
extrapolate the aIC₅₀. Experiment was run in
triplicate (n = 3). The selectivity index was
defined as the ratio of the concentration of drug
required to kill 50% of the cells in normal cells,
divided by the concentration required to exert the
same efficacy in tumor cells. Cells were seeded in
a 96-well plate according to their optimal cell
densities (5.000-10.000 cells/well). Therefore, a
SI below 1 is considered not selective, while the
higher the index, the better the selectivity.
When SI is at least 2, these compounds could
potentially have a more beneficial therapeutic
window. Values are calculated for selectivity
towards HME-1 cells, * towards HUVEC cells (SI =
^{HUVEC}IC₅₀ / ^{cancer}IC₅₀) or **towards hiPSCs (iAstro, SI =
^{iAstro}IC₅₀ / ^{cancer}IC₅₀).

| SI = ^{normal}IC₅₀ / ^{cancer}IC₅₀ | GBM | | Melanoma | TNBC |
|---|---|---|---|---|
| Compound | A-172 | U-87 MG | A2058 | MDA-MB-231 |
| MMAE | 0.3 | 0.4 | 1.1 | 0.4 |
| | *1.8 | *2.5 | *7.4 | *2.8 |
| | **0.6 | **0.9 | **nd | **nd |
| PhAc-ALLP-PABC-MMAE | 0.1 | 0.1 | 0.3 | 0.2 |
| | *1.9 | *2.4 | *6.2 | *3.8 |
| | **>500 | **>500 | **nd | **nd |
| PhAc-ALLP-Dox | 30.5 | 128.5 | 298.2 | 20.9 |
| | *34.4 | *144.9 | *336.1 | *23.6 |
| | **15.8 | **14.1 | **nd | **nd |

### Example 4. Materials and Methods

TABLE 7
Overview of drugs and prodrugs used

| Compound | Purity (%) | MW (g/mol) |
|---|---|---|
| Doxorubicin HCl | 99.8 | 580 |
| PhAc-APKP-Dox | 97.8 | 1058.39 |
| PhAc-ALLP-Dox | 95.1 | 1059.41 |
| MMAE | 99.1 | 717.99 |
| PhAc-ALLP-PABC-MMAE | 98.8 | 1385.65 |

### Treatment and Drugs.

Doxorubicin-HCl was acquired from LC-Labs (D-4000-500 mg), while MMAE and the prodrug compounds were synthetized by WuXi AppTec (China). Stock solutions of 10 mM were dissolved in DMSO or in H₂O (PhAc-ALLP-PABC-MMAE) and stored at −20° C. till just prior to use.

### In Vitro Potency, Maximal Efficacy and Selectivity Index

All commercial cell lines were purchased at ATCC (LGC Standards SARL, France). Cells were seeded in 96-well plates according to their optimal seeding density, varying between 5.000 and 10.000 cells/well. After attachment overnight, 10-point serial titrations (1:5) were prepared in their equivalent complete media as recommended by ATCC, starting from the 10 mM stock solutions. For doxorubicin comprising prodrugs dilutions started from 100 μM, while for the parent compound doxorubicin dilutions started at 10 μM. In case of the much more potent MMAE and PhAc-ALLP-PABC-MMAE, similar dilution series were prepared in media, starting at 500 nM. Seventy-two hours later, compounds were removed together with the supernatants and cells were washed once with PBS to remove excess drugs.

The WST-1 assay for cell proliferation and viability (Roche, Switzerland) was performed according to manufacturer's protocol. Absorbances were measured at 4 hrs using a Perkin Elmer Ensight multiplate reader, equipped with Kaleido 2.0 software (Perkin Elmer, USA). Cell viability was expressed as a percentage compared to non-treated cells. Absolute IC₅₀ values were extrapolated from a non-linear fitting following the Sigmoidal-4PL regression method using Graphpad Prism 7.0. Likewise, maximal efficacy was determined as 100% minus the cell viability determined when cells are incubated in the presence of a compound. For instance, if 20% of the cells survive incubation in the presence of a compound, then the maximal efficacy of that compound is 100%-20%=80%.

Selectivity indices (SI) were defined as the ratio of the concentration of drug required to kill 50% of the cells in normal (control, non-cancer) cells, divided by the concentration required to exert the same efficacy in tumor cells: SI=IC₅₀ (control cell)/IC₅₀ (cancer cell), or SI=^{HME-1}IC₅₀/cancer IC₅₀.

Human Induced-Pluripotent Stem Cell Derived Astrocytes (iAstro™)

hiPSC-derived astrocytes were purchased from Tempo Biosciences (iAstro™). Prior to use, six-well plates were coated with 1 ml GFR Matrigel (1:100˜0.1 mg/ml, Corning #356231) and allowed to polymerize at 37° C. overnight. Three days before assaying, cells were defrosted and seeded onto the GFR Matrigel in iAstro medium, and allowed to recover from defrosting for 48 hrs. The recovery was aided using RevitaCell™ Supplement during the first 24 hrs. After morphological inspection, cells were transferred to 96-well plates and 4-well microscopy vessels (Millipore), coated with Poly-L-lysine (50 μg/ml, P2533, Sigma Aldrich) and mouse laminin (4 ng/ml, L2020, Sigma Aldrich), using StemPro Accutase reagent (Invitrogen). Following overnight recovery, iAstro's were exposed to the same treatment protocol described above.

### Example 5. In Vivo Activity of PhAc-ALLP-Dox on Colorectal Cancer

The in vivo efficacy of PhAc-ALLP-Dox was tested in a mouse model of colorectal cancer. LS-174T colorectal tumor cells were xenografted in Nude NMRI mice. In total, 30 adult (9-10 weeks old) nude female NMRI mice (Janvier, France) were subjected to subcutaneous (SC) tumor cell grafting in the right flank. In total, 2×10\hat{ }6 LS-174T cells resuspended in PBS were injected in 200 uL final volume. As soon as the tumors were palpable and reached the volume of 200 mm3 the treatment was started. At day zero, mice were randomly divided into 3 groups, based on the tumor volume, in order to have the following experimental sub-cohorts:
control group receiving the vehicle (0.9% NaCl) with a dose of 5 ml/kg, intravenously (iv), twice per week (Q2W);
PhAc-ALLP-Dox (10 mg/kg iv, Q2W); and
PhAc-ALLP-Dox (30 mg/kg iv, Q2W).

Treatments were performed twice per week for a total of four tail vein administrations/injections (on days 1, 4, 7 and 10). One extra week of observation followed the active treatment phase. During the experiment, tumor volume was measured two times a week and was assessed three-dimensionally with a digital caliper (Mitutoyo, Illinois) using the following formula:

V=4/3π×[(d/2)²×(D/2)], where d is the minor tumor axis and D is the major tumor axis.

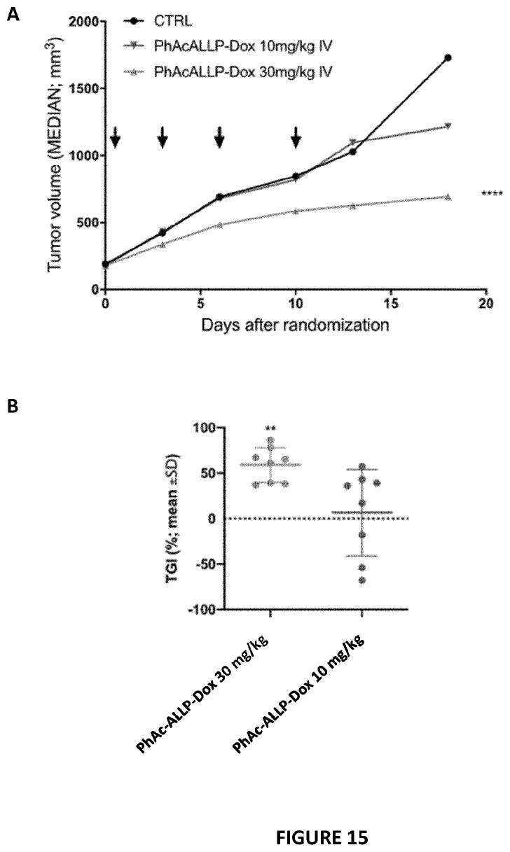

The results are depicted in FIG. 15A. Importantly, mice did tolerate both doses of the prodrug and the treatment arm which received 30 mg/kg PhAc-ALLP-Dox significantly reduced the tumor volume when compared to the untreated control group.

Tumor growth inhibition (TGI) expressed as percentage vs control was calculated as follows: % TGI=(1−{Tt/T0/Ct/C0}/1−{C0/Ct})×100 where Tt and T0 are the individual tumor volume of treated mouse X at times t and 0 respectively, Ct and C0 are the mean tumor volume of the control group at times t and 0 respectively. As indicated in FIG. 15B, in the cohort treated with 30 mg/kg PhAc-ALLP-Dox, a TGI of approximately 60% was obtained.

No overt sign of toxicity nor significant reduction of body weight or alteration in blood count was observed in dosed mice.

### Example 6. In Vivo Activity of PhAc-ALLP-PABC-MMAE on Melanoma

The in vivo efficacy of PhAc-ALLP-PABC-MMAE was tested in a mouse melanoma model. A2058 melanoma cells were subcutaneously (SC) implanted in nude NMRI mice. In total, 36 adult (9-10 weeks old) nude female NMRI mice (Janvier, France) were subjected to SC tumor cell grafting in the right flank. In total, 3×10⁶ A2058 cells resuspended in PBS plus Matrigel (1:1) were injected in 200 uL final volume. As soon as the tumors were palpable and reached the volume of 200 mm3 the treatment was started. At day zero, mice were randomly divided into 4 groups, based on the tumor volume, in order to have the following experimental sub-cohorts:
control group receiving the vehicle (PBS pH7.2) with a dose of 5 ml/kg, intravenously (iv), once per week (QW);
PhAc-ALLP-PABC-MMAE (2 mg/kg iv, QW);
PhAc-ALLP-PABC-MMAE (4 mg/kg iv, QW);
MMAE (0.9 mg/kg iv, QW).

Treatments were performed once per week for a total of four tail vein administrations/injections (on days 1, 7, 14 and 21). Two extra weeks of observation followed the active treatment phase. During the experiment, tumor volume was measured two times a week and was assessed three-dimensionally with a digital caliper (Mitutoyo, Illinois) using the following formula:

V=4/3π×[(d/2)²×(D/2)], where d is the minor tumor axis and D is the major tumor axis.

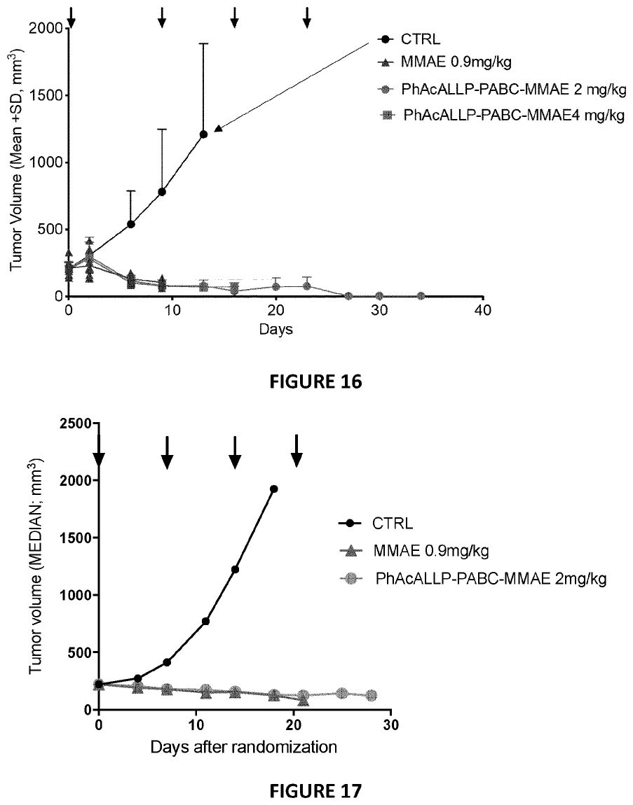

Results are given in FIG. 16. The cohort which received 2 mg/kg PhAc-ALLP-PABC-MMAE importantly reduced the tumor volume when compared to the untreated control group. Complete response was observed until day 34 without any hint of relapse or macroscopical sign of toxicity. Mice dosed with higher concentration of PhAc-ALLP-PABC-MMAE, despite important reduction of tumor growth, were sacrificed at day 21 because significant reduction of body weight (>20%).

### Example 7. In Vivo Activity of PhAc-ALLP-PABC-MMAE on Glioblastoma (GBM)

The in vivo efficacy of PhAc-ALLP-PABC-MMAE was tested in a mouse GBM model. U87 MG glioblastoma cells were subcutaneously (SC) implanted in nude NMRI mice. In total, 24 adult (9-10 weeks old) nude female NMRI mice (Janvier, France) were subjected to SC tumor cell grafting in the right flank. In total, 5×10⁶ U87 MG cells resuspended in PBS plus Matrigel (1:1) were injected in 200 uL final volume. As soon as the tumors were palpable and reached the volume of 200 mm3 the treatment was started. At day zero, mice were randomly divided into 3 groups, based on the tumor volume, in order to have the following experimental sub-cohorts:
control group receiving the vehicle (PBS pH7.2) with a dose of 5 ml/kg, intravenously (iv), once per week (QW);
PhAc-ALLP-PABC-MMAE (2 mg/kg iv, QW);
MMAE (0.9 mg/kg iv, QW).

Treatments were performed once per week for a total of four tail vein administrations/injections (on days 1, 7, 14 and 21). One extra week of observation followed the active treatment phase. During the experiment, tumor volume was measured two times a week and was assessed three-dimensionally with a digital caliper (Mitutoyo, Illinois) using the following formula:

V=4/3π×[(d/2)²×(D/2)], where d is the minor tumor axis and D is the major tumor axis.

Results are given in FIG. 17. The cohort which received 2 mg/kg PhAc-ALLP-PABC-MMAE importantly reduced the tumor volume when compared to the untreated control group without any hint of macroscopical sign of toxicity. On the contrary, mice dosed with 0.9 mg/kg MMAE, despite important reduction of tumor growth, were sacrificed at day 21 because significant reduction of body weight (>20%).

### Example 8. In Vivo Activity of PhAc-ALLP-Dox on Glioblastoma (GBM)

The in vivo efficacy of PhAc-ALLP-Dox was tested in a mouse GBM model. U87 MG glioblastoma cells were subcutaneously (SC) implanted in nude NMRI mice. In total, 32 adult (9-10 weeks old) nude female NMRI mice (Janvier, France) were subjected to SC tumor cell grafting in the right flank. In total, 5×10⁶ U87 MG cells resuspended in PBS plus Matrigel (1:1) were injected in 200 uL final volume. As soon as the tumors were palpable and reached the volume of 200 mm3 the treatment was started. At day zero, mice were randomly divided into 4 groups, based on the tumor volume, in order to have the following experimental sub-cohorts:
control group receiving the vehicle (PBS pH7.2) with a dose of 5 ml/kg, intravenously (iv), once per week (QW);
PhAc-ALLP-Dox (30 mg/kg iv, QW);
PhAc-ALGP-Dox (154 mg/kg iv, QW);
Dox (5 mg/kg iv, QW).

Treatments were performed once per week for a total of four tail vein administrations/injections (on days 1, 7, 14 and 21). One extra week of observation followed the active treatment phase. During the experiment, tumor volume was measured two times a week and was assessed three-dimensionally with a digital caliper (Mitutoyo, Illinois) using the following formula:

V=4/3π×[(d/2)²×(D/2)], where d is the minor tumor axis and D is the major tumor axis.

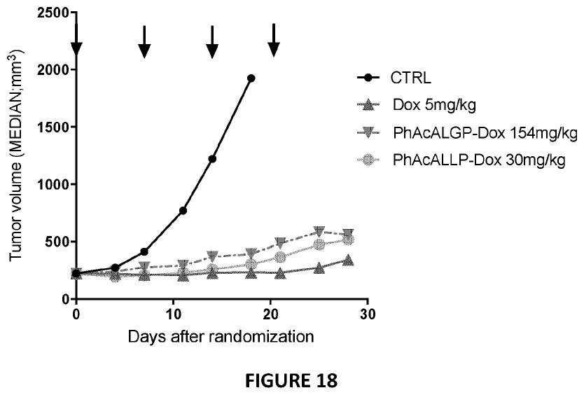

Results are given in FIG. 18. The cohort which received 30 mg/kg PhAc-ALLP-Dox importantly reduced the tumor volume when compared to the untreated control group. Mice dosed with 5 mg/kg Dox or with 154 mg/kg PhAc-ALGP-Dox significantly reduced tumor growth similarly to PhAc-ALLP-Dox. None of the treated cohorts revealed any sign of macroscopic toxicity nor important body weight loss (>20%).

1. A compound having the general structure C-OP-D, wherein:
C is a capping group;
OP is the tetrapeptidic moiety ALLP (SEQ ID NO:1) or APKP (SEQ ID NO:2);
D is a drug;
or a pharmaceutically acceptable salt of said compound, a pharmaceutically acceptable crystal or co-crystal comprising said compound, or a pharmaceutically acceptable polymorph, isomer, or amorphous form of said compound.

2. The compound, salt, crystal, co-crystal, polymorph or isomer according to claim 1 wherein D is a cytotoxic drug, a cytostatic drug, or is an anti-cancer drug.

3. The compound, salt, crystal, co-crystal, polymorph, isomer or amorphous form according to claim 1 wherein the linkage between OP and D is direct or is indirect via a linker or spacing group.

4. The compound, salt, crystal, co-crystal, polymorph, isomer or amorphous form according to claim 3 wherein said linker or spacing group is a self-eliminating linker or spacing group.

5. The compound, salt, crystal, co-crystal, polymorph, isomer or amorphous form according to claim 1 wherein the linkage between C and OP is direct, or is indirect via a linker or spacing group.

6. The compound, salt, crystal, co-crystal, polymorph, isomer or amorphous form according to claim 1 further complexed with a macrocyclic moiety.

7. A composition comprising the compound, salt, crystal, co-crystal, polymorph, isomer or amorphous form according to claim 1.

8. The composition according to claim 7 further comprising at least one of a pharmaceutically acceptable solvent, diluent or carrier.

9. The compound, salt, crystal, co-crystal, polymorph, isomer or amorphous form according to claim 1 for use as a medicament.

10. The compound, salt, crystal, co-crystal, polymorph, isomer or amorphous form according to claim 1 for use in the treatment of a cancer.

11. The compound, salt, crystal, co-crystal, polymorph, isomer or amorphous form according to claim 10, wherein said treatment of cancer is a combination chemotherapy treatment or a combined modality chemotherapy treatment.

12. A method for producing a compound according to claim 1, said method comprising the steps of: linking the drug D, the tetrapeptidic moiety OP, and the capping group C; wherein the linking of D, OP and C is resulting in the compound C-OP-D, and wherein the linking between drug D and tetrapeptidic moiety OP is direct or via a linker or spacing group and/or the linking between the capping group C and the tetrapeptidic moiety OP is direct or via a linker or spacing group.

13. The method for producing a compound according to claim 12 further comprising the step of purifying the compound C-OP-D.

14. The method for producing a compound according to claim 12 further comprising forming a salt, amorphous form, crystal or co-crystal of the compound C-OP-D.

15. A kit comprising a container comprising the compound, salt, crystal, co-crystal, polymorph, isomer or amorphous form according to claim 1.


SEQUENCE LISTING (3 sequences)

SEQ ID NO: 1 - protein, 4 aa, artificial sequence
  synthetic oligopeptide
ALLP

SEQ ID NO: 2 - protein, 4 aa, artificial sequence
  synthetic oligopeptide
APKP

SEQ ID NO: 3 - protein, 4 aa, artificial sequence
  synthetic oligopeptide
ALGP